(19) United States
(12) Patent Application Publication
(10) Pub. No.: US 2022/0309415 A1
(43) Pub. Date: Sep. 29, 2022

(54) INFORMATION PROCESSING APPARATUS, CONTROL METHOD THEREFOR, PROGRAM, AND SYSTEM

(71) Applicant: CANON KABUSHIKI KAISHA, Tokyo (JP)

(72) Inventor: Motohiro Furusawa, Shizuoka (JP)

(21) Appl. No.: 17/613,195

(22) PCT Filed: Jul. 10, 2020
(86) PCT No.: PCT/JP2020/027067

§ 371 Date: Nov. 22, 2021

(30) Foreign Application Priority Data
Jul. 16, 2019 (JP) 2019-131399

(51) Int. Cl. G06Q 10/06; A61L 2/28; A61L 2/24; G16H 40/20

(52) U.S. Cl. CPC G06Q 10/0633; A61L 2/28; A61L 2/24; G16H 40/20; A61L 2202/14; A61L 2202/24

(57) ABSTRACT

A system for evaluating quality of a sterilization workflow concerning sterilization of a sterilization target, comprises registration means for registering, for each base, basic information concerning the base where the sterilization workflow is performed; accumulation means for acquiring, for each base, accumulation information indicating a result of a work associated with the sterilization workflow, and accumulate the accumulation information; and evaluation means for evaluating, in accordance with an evaluation target and an evaluation item designated by a user of the system, at least one evaluation item concerning at least one stage of the sterilization workflow using basic information concerning the evaluation target and the accumulation information.

### TECHNICAL FIELD

The present invention relates to an information processing apparatus concerning sterilization processing of a sterilization target, a control method for the information processing apparatus, a program, and a system.

### BACKGROUND ART

In a medical institution, sterilization processing of a sterilization target such as a medical instrument is performed as infection control. At this time, to determine the achievement degree of the sterilization processing, a so-called sterilization indicator such as a chemical indicator (to be referred to as a CI hereinafter), a biological indicator (to be referred to as a BI hereinafter), or a physical indicator is used. The CI includes a discoloration region that is discolored in accordance with the achievement degree of a condition necessary for sterilization processing by a sterilant (steam, hydrogen peroxide, or the like). It is possible to determine the achievement degree of the sterilization processing based on the color of the discoloration region of the CI after the sterilization processing. The BI includes indicator bacteria having strong resistance against a sterilant, and indicator bacteria die out in accordance with the achievement degree of a condition necessary for sterilization processing. After the sterilization processing, the BI is cultivated to confirm the existence of the indicator bacteria, thereby making it possible to determine the achievement degree of the sterilization processing. The physical indicator is the operation record of a sterilization processing apparatus, and an operation status is monitored and recorded for each operation. This makes it possible to determine whether the target operation could be achieved. PTL 1 proposes a management apparatus that manages the record of sterilization processing. More specifically, the management apparatus records the date/time of sterilization processing for a sterilization target, a sterilization condition, and the determination result of a CI.

### CITATION LIST

### Patent Literature

[PTL 1] Japanese Patent No. 3414291

### SUMMARY OF INVENTION

However, the above-described conventional technique has the following problems. For example, the above-described conventional technique simply records parameters concerning sterilization processing but does not sufficiently examine improvement of the sterilization processing and a system by a quality evaluation of the sterilization processing and its feedback using the record of the parameters. However, the sterilization processing includes various stages and is achieved by involving many people, and a malfunction may occur in each stage due to various factors such as an artificial mistake and a failure of an apparatus. Depending on the malfunction, a problem may arise in safety of a sterilization target, and it is demanded to reduce such malfunction as much as possible. In addition, in each medical institution and its related facilities, sterilization processing is performed by various methods, and objective and quantitative evaluations of them are also required. For example, the medical institution or the like can use the evaluations to introduce a better sterilization processing system or to improve a sterilization processing system to a better one. Furthermore, a patient can use the evaluations as criteria to select a medical institution or the like.

The present invention has been made in consideration of at least one of the above-described problems, and provides, for example, a mechanism of preferably performing a quality evaluation in a sterilization processing system. For another purpose, the present invention preferably provides the quality evaluation and related parameters, as needed. The present invention also provides quality evaluations and related parameters in a plurality of sterilization processing systems so as to allow comparison between them.

One aspect of the present invention provides a system for evaluating quality of a sterilization workflow concerning sterilization of a sterilization target, the sterilization workflow having a plurality of stages including at least one of an assembly stage of grouping at least one sterilization target, a packaging stage of forming a set by packaging the at least one sterilization target, a sterilization stage of performing sterilization processing of the set by a sterilization processing apparatus, a storage stage of storing the set having undergone the sterilization processing in a predetermined storage place, a delivery stage of moving the set from the predetermined storage place to a place where the set is used, a use stage of using the at least one sterilization target included in the set by determining an achievement degree of the sterilization processing for the set, a collection stage of collecting the sterilization target used, and a cleaning stage of cleaning the collected sterilization target, the system comprising: registration means for registering, for each base, basic information concerning the base where the sterilization workflow is performed; accumulation means for acquiring, for each base, accumulation information indicating a result of a work associated with the sterilization workflow, and accumulate the accumulation information; and evaluation means for evaluate, in accordance with an evaluation target and an evaluation item designated by a user of the system, at least one evaluation item concerning at least one stage of the sterilization workflow using basic information concerning the evaluation target and the accumulation information.

Another aspect of the present invention provides an information processing apparatus for evaluating quality of a sterilization workflow concerning sterilization of a sterilization target, the sterilization workflow having a plurality of stages including at least one of an assembly stage of grouping at least one sterilization target, a packaging stage of forming a set by packaging the at least one sterilization target, a sterilization stage of performing sterilization processing of the set by a sterilization processing apparatus, a storage stage of storing the set having undergone the sterilization processing in a predetermined storage place, a delivery stage of moving the set from the predetermined storage place to a place where the set is used, a use stage of using the at least one sterilization target included in the set by determining an achievement degree of the sterilization processing for the set, a collection stage of collecting the sterilization target used, and a cleaning stage of cleaning the collected sterilization target, the apparatus comprising: registration means for acquiring, for each base, from an external apparatus, basic information concerning the base where the sterilization workflow is performed, and register the acquired basic information; accumulation means for acquiring, for each base, from an external apparatus, accumulation information indicating a result of a work associated with the sterilization workflow, and accumulate the acquired accumulation information; evaluation means for evaluating, in accordance with an evaluation target and an evaluation item designated by a user via an external apparatus, at least one evaluation item concerning at least one stage of the sterilization workflow using basic information concerning the evaluation target and the accumulation information; and providing means for providing information for visualizing an evaluation result of the evaluation means to an external apparatus used by the user.

Still another aspect of the present invention provides a control method for an information processing apparatus for evaluating quality of a sterilization workflow concerning sterilization of a sterilization target, the sterilization workflow having a plurality of stages including at least one of an assembly stage of grouping at least one sterilization target, a packaging stage of forming a set by packaging the at least one sterilization target, a sterilization stage of performing sterilization processing of the set by a sterilization processing apparatus, a storage stage of storing the set having undergone the sterilization processing in a predetermined storage place, a delivery stage of moving the set from the predetermined storage place to a place where the set is used, a use stage of using the at least one sterilization target included in the set by determining an achievement degree of the sterilization processing for the set, a collection stage of collecting the sterilization target used, and a cleaning stage of cleaning the collected sterilization target, the method comprising: acquiring, for each base, from an external apparatus, basic information concerning the base where the sterilization workflow is performed, and registering the acquired basic information; acquiring, for each base, from an external apparatus, accumulation information indicating a result of a work associated with the sterilization workflow, and accumulating the acquired accumulation information; evaluating, in accordance with an evaluation target and an evaluation item designated by a user via an external apparatus, at least one evaluation item concerning at least one stage of the sterilization workflow using basic information concerning the evaluation target and the accumulation information; and providing information for visualizing an evaluation result of the evaluation to an external apparatus used by the user.

Yet still another aspect of the present invention provides a program for causing a computer to execute each step of a control method for an information processing apparatus for evaluating quality of a sterilization workflow concerning sterilization of a sterilization target, the sterilization workflow having a plurality of stages including at least one of an assembly stage of grouping at least one sterilization target, a packaging stage of forming a set by packaging the at least one sterilization target, a sterilization stage of performing sterilization processing of the set by a sterilization processing apparatus, a storage stage of storing the set having undergone the sterilization processing in a predetermined storage place, a delivery stage of moving the set from the predetermined storage place to a place where the set is used, a use stage of using the at least one sterilization target included in the set by determining an achievement degree of the sterilization processing for the set, a collection stage of collecting the sterilization target used, and a cleaning stage of cleaning the collected sterilization target, the method comprising: acquiring, for each base, from an external apparatus, basic information concerning the base where the sterilization workflow is performed, and registering the acquired basic information; acquiring, for each base, from an external apparatus, accumulation information indicating a result of a work associated with the sterilization workflow, and accumulating the acquired accumulation information; evaluating, in accordance with an evaluation target and an evaluation item designated by a user via an external apparatus, at least one evaluation item concerning at least one stage of the sterilization workflow using basic information concerning the evaluation target and the accumulation information; and providing information for visualizing an evaluation result of the evaluation to an external apparatus used by the user.

Still yet another aspect of the present invention provides a system comprising a server configured to evaluate quality of a sterilization workflow concerning sterilization of a sterilization target and a user terminal configured to use a service provided by the server, the sterilization workflow having a plurality of stages including at least one of an assembly stage of grouping at least one sterilization target, a packaging stage of forming a set by packaging the at least one sterilization target, a sterilization stage of performing sterilization processing of the set by a sterilization processing apparatus, a storage stage of storing the set having undergone the sterilization processing in a predetermined storage place, a delivery stage of moving the set from the predetermined storage place to a place where the set is used, a use stage of using the at least one sterilization target included in the set by determining an achievement degree of the sterilization processing for the set, a collection stage of collecting the sterilization target used, and a cleaning stage of cleaning the collected sterilization target, the server comprising registration means for acquiring, for each base, from an external apparatus, basic information concerning the base where the sterilization workflow is performed, and register the acquired basic information, accumulation means for acquiring, for each base, from an external apparatus, accumulation information indicating a result of a work associated with the sterilization workflow, and accumulate the acquired accumulation information, evaluation means for evaluating, in accordance with an evaluation target and an evaluation item designated by a user via the user terminal, at least one evaluation item concerning at least one stage of the sterilization workflow using basic information concerning the evaluation target and the accumulation information, and providing means for providing information for visualizing an evaluation result of the evaluation means to the user terminal, and the user terminal comprising designation means for designating the evaluation target and the evaluation item, and display means for display, on display means, the information provided by the providing means.

Yet still another aspect of the present invention provides an information processing apparatus comprising: input means for inputting an evaluation target for which at least one evaluation item concerning at least one stage of a sterilization workflow is evaluated; selection means for selecting at least one evaluation item that can be evaluated with respect to the evaluation target input via the input means; and display control means for displaying, on display means, an evaluation result of the at least one evaluation item selected via the selection means.

According to the present invention, it is possible to preferably perform a quality evaluation in a sterilization processing system. Furthermore, it is possible to preferably provide the quality evaluation and related parameters, as needed. It is also possible to provide quality evaluations and related parameters in a plurality of sterilization processing systems so as to allow comparison between them.

Further features of the present invention will become apparent from the following description of exemplary embodiments (with reference to the attached drawings).

### BRIEF DESCRIPTION OF DRAWINGS

FIG. 1 is a view showing a sterilization workflow according to an embodiment.

FIG. 2A is a block diagram showing the arrangement of a system of the sterilization workflow according to the embodiment.

FIG. 2B is a block diagram showing examples of the arrangements of respective apparatuses according to the embodiment.

FIG. 2C is a block diagram showing the functional components of each server according to the embodiment.

FIG. 3 is a view showing a CI according to the embodiment

FIG. 4 is a sectional view of a measurement apparatus according to the embodiment.

FIG. 5 is a view showing a BI according to the embodiment.

FIG. 6A show a table each showing an example of management information in a base server 14 according to the embodiment.

FIG. 6B show a table each showing an example of management information in a base server 14 according to the embodiment.

FIG. 6C show a table each showing an example of management information in a base server 14 according to the embodiment.

FIG. 7 is a table showing a type determination table according to the embodiment.

FIG. 8 is a table showing an example of basic registration information registered in an evaluation analysis server 16 according to the embodiment.

FIG. 9 is a table showing an example of accumulation information accumulated in the evaluation analysis server 16 according to the embodiment.

FIG. 10 shows evaluation tables according to the embodiment.

FIG. 11 is a view showing a UI of an individual evaluation report according to the embodiment.

FIG. 12 is a view showing output of the individual evaluation report according to the embodiment.

FIG. 13 is a view showing a line chart of the individual evaluation report according to the embodiment.

FIG. 14A is a view showing a radar chart of the individual evaluation report according to the embodiment.

FIG. 14B is a view showing a stage-specific radar chart according to the embodiment.

FIG. 15A is a flowchart illustrating a processing procedure of the individual evaluation report according to the embodiment.

FIG. 15B is a flowchart illustrating a subroutine of individual evaluation processing according to the embodiment.

FIG. 16 is a view showing a UI of a comparison evaluation report according to another embodiment.

FIG. 17 is a view showing output of the comparison evaluation report according to the other embodiment.

FIG. 18A is a flowchart illustrating a processing procedure of the comparison evaluation report according to the other embodiment.

FIG. 18B is a flowchart illustrating a subroutine of comparison evaluation processing according to the other embodiment.

FIG. 19 is a view showing a UI of patient-related information according to still another embodiment.

FIG. 20 is a flowchart illustrating a processing procedure of the patient-related information according to the embodiment shown in FIG. 19.

### DESCRIPTION OF EMBODIMENTS

Hereinafter, embodiments will be described in detail with reference to the attached drawings. Note, the following embodiments are not intended to limit the scope of the claimed invention. Multiple features are described in the embodiments, but limitation is not made an invention that requires all such features, and multiple such features may be combined as appropriate. Furthermore, in the attached drawings, the same reference numerals are given to the same or similar configurations, and redundant description thereof is omitted.

### First Embodiment

Sterilization Workflow

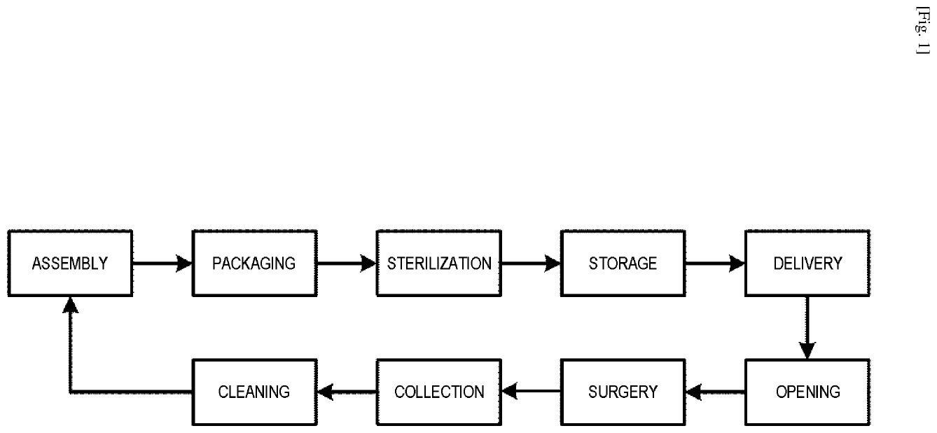

The first embodiment of the present invention will be described below. FIG. 1 shows a sterilization workflow concerning sterilization processing. In this embodiment, the sterilization workflow indicates a series of stages performed with respect to sterilization processing for a sterilization target. Stages to be described below are merely examples, and are not intended to limit the present invention. Other stages and the like may be included, and some of the stages to be described below may not be included due to the type of sterilization processing or various factors. For example, if resterilization is performed without using a set having undergone sterilization processing, a cleaning stage, an assembly stage, and the like may not be included depending on a factor.

The assembly stage is a stage of grouping one or more sterilization targets (medical instruments and the like) in a sterilization station. Note that the number of sterilization targets included in one group may be one. Grouping can be performed based on the correspondence between a surgery in which the sterilization target is used and a sterilization processing apparatus used for sterilization processing of the sterilization target. For example, sterilization targets that are used in the same surgery and undergo sterilization processing by the same sterilization processing apparatus are grouped in the same group. A packaging stage is a stage of forming one set by packaging one or more sterilization targets of one group and a CI (first indicator) by one packaging member in the sterilization station. An example in which the CI is enclosed will be described here. However, no CI may be enclosed (in this case, only a PCD CI is used), or a BI (second indicator) may be enclosed in addition to or instead of the CI. In a hospital or the like, a plurality of sterilization targets of the same type are normally used, and thus a plurality of sets including the sterilization targets of the same type are formed. The sets including the sterilization targets of the same type will be referred to as sets of the same type hereinafter. Conversely, if the types of two sets are different, at least one of the sterilization targets included in one of the two sets is different from that included in the other set. The CI packaged in the set in the packaging stage is decided in accordance with the type of the sterilization processing apparatus that performs sterilization processing for the set. A sterilization stage is a stage of performing sterilization processing for the packaged set by the sterilization processing apparatus.

A storage stage is a stage of moving the set having undergone the sterilization processing in the sterilization stage to a storage to store it. A delivery stage is a stage of moving, from the storage to a use place such as an operating room, the set to be used in a surgery or the like. An opening stage is a stage of opening the package of the set moved to the use place. A surgery stage is also a use stage of using the sterilization target. Note that in one or both (to be referred to as the use stage hereinafter) of the opening stage and the surgery stage, the achievement degree of the sterilization processing is determined using the CI enclosed in the set before using the sterilization target. Only if it can be determined that the achievement degree of the sterilization processing by the CI satisfies a predetermined criterion, the sterilization target is actually used in the surgery stage. A collection stage is a stage of moving the sterilization target to the sterilization station after the use of the sterilization target. The cleaning stage is a stage of cleaning the sterilization target in the sterilization station. After cleaning the set in the cleaning stage, the set is sent to the assembly stage again. Note that the above stages of the sterilization workflow according to this embodiment are merely examples, and the sterilization workflow of the present invention is not limited to that including the above stages. For example, the sterilization workflow may include a stage other than the above stages. Furthermore, the sterilization workflow may be obtained by dividing at least one of the above stages into a plurality of stages. The sterilization workflow may be obtained by integrating at least two of the above stages into one stage.

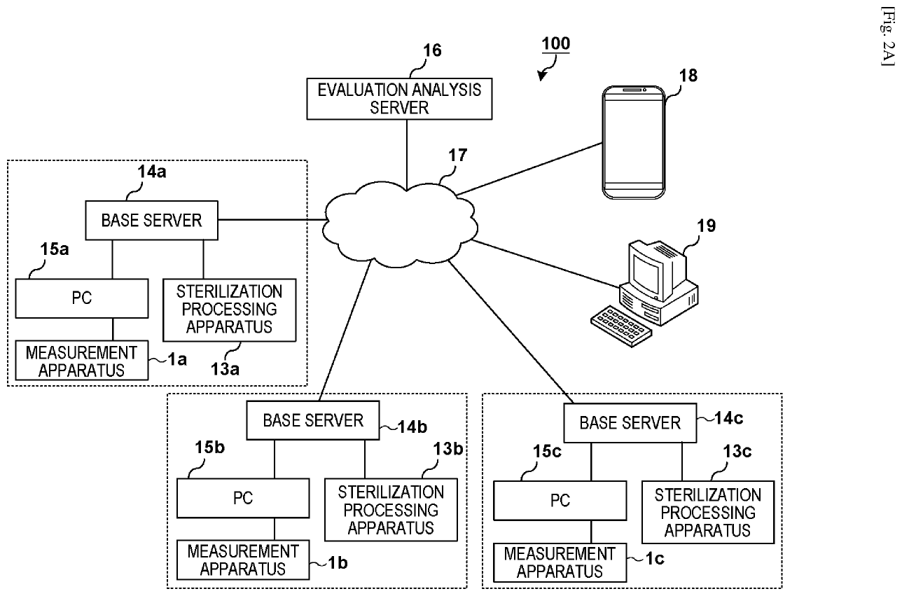

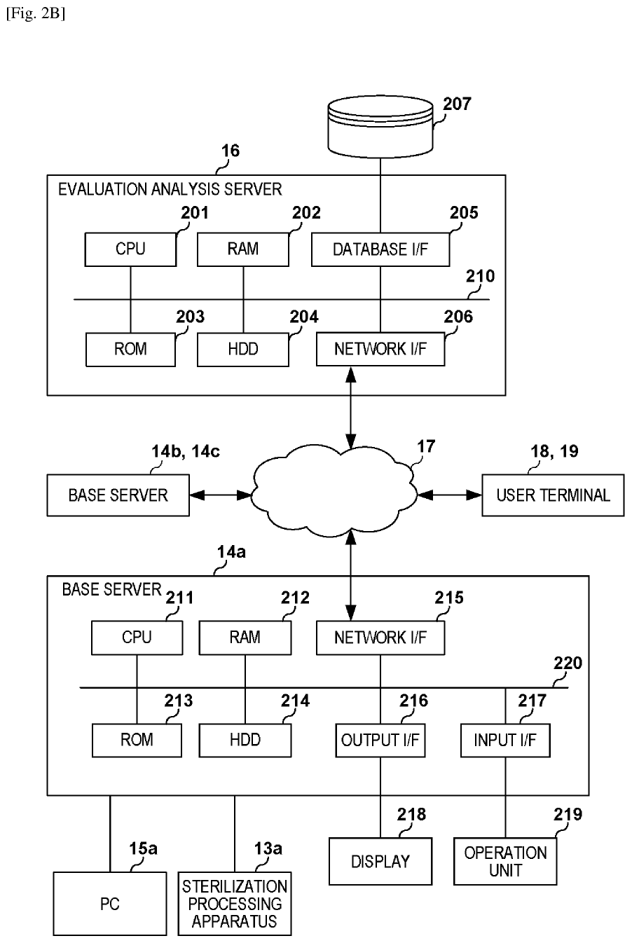

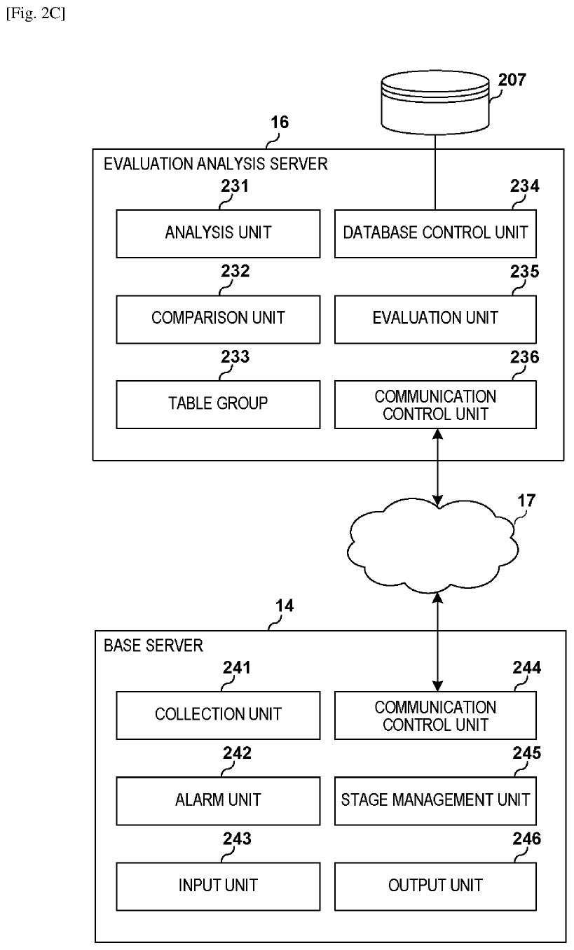

System Arrangement FIG. 2A shows an example of the arrangement of a system 100 that evaluates the above sterilization workflow. In one base such as a medical institution, at least a base server 14, a sterilization processing apparatus 13, a measurement apparatus 1, and a PC 15 are arranged. These apparatuses are installed in each base, and a different alphabet (a, b, or c) is appended to each reference numeral for each base. Note that when explaining the same control and arrangement of each apparatus which is installed in each base, only the reference numeral is described without appending the alphabet. In this system, a plurality of base systems are provided, and are communicably connected to an evaluation analysis server (information processing apparatus) 16 via a network 17. The base server 14, the PC 15, and the sterilization processing apparatus 13 are communicably interconnected via a LAN in each base. Furthermore, a user terminal 18 as a moving device such as a smartphone or a tablet and a user terminal 19 such as a desktop PC or a notebook PC are connected to the evaluation analysis server 16 via the network 17. Each of these user terminals 18 and 19 is a device of a patient, and the patient who used or will use the base such as a hospital can access information of an evaluation about the base, information of a sterilization target used for the patient, and the like. Note that the base server 14, the PC 15, and the user terminals 18 and 19 also function as user terminals used by the user of this system.

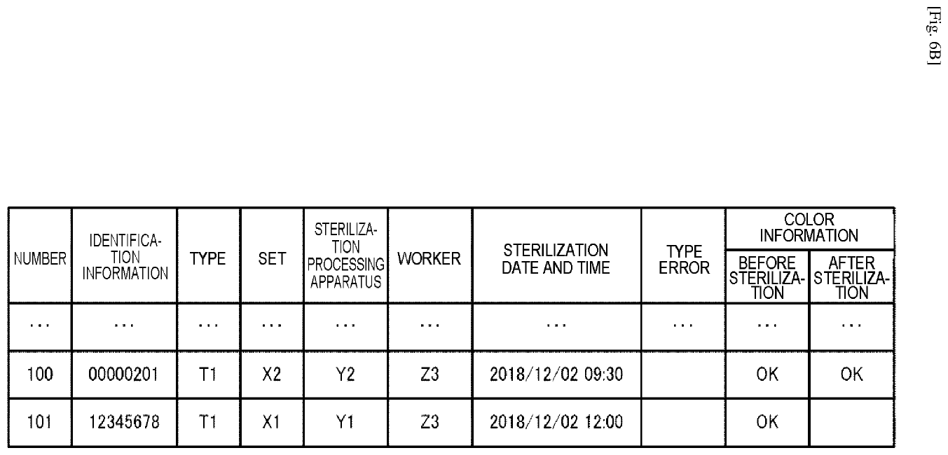

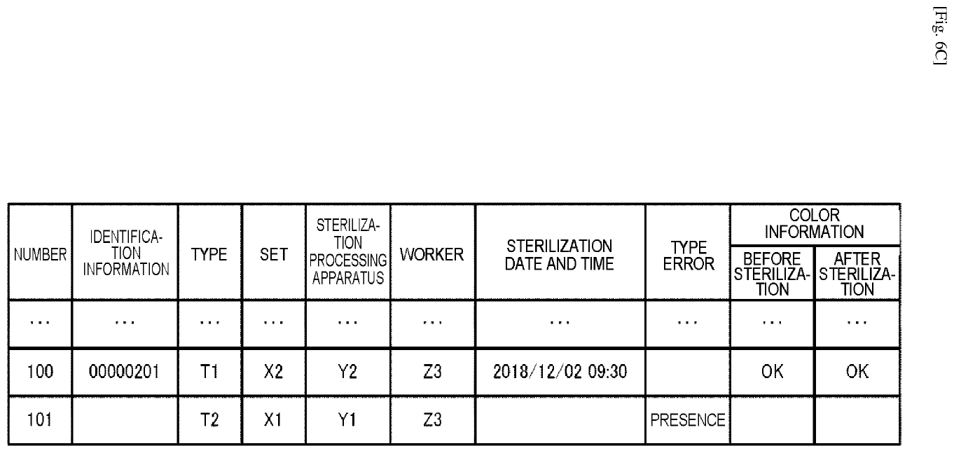

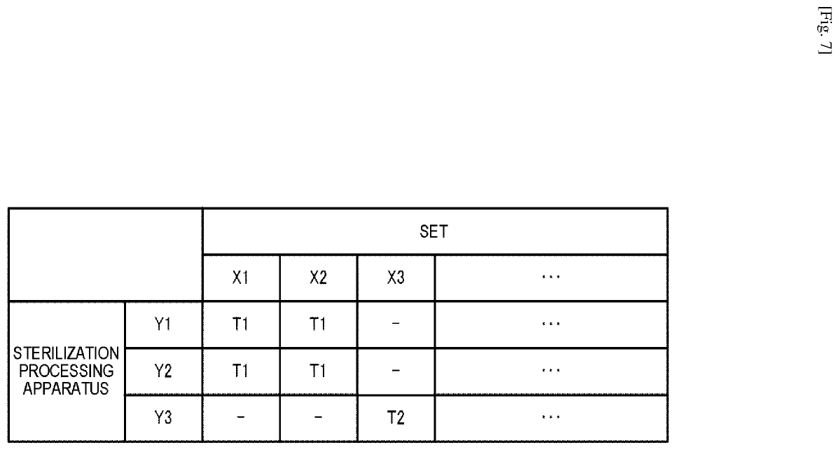

The evaluation analysis server 16 accumulates information from each base server 14, for example, base-related information for each base, and management information and history information acquired at any time, and then evaluates and analyzes the sterilization processing system of each base in accordance with the accumulation information. The management information indicates information concerning a sterilization indicator (in this example, mainly a CI), as will be described later with reference to FIGS. 6A-6C. The history information indicates information such as information of various types of indicators such as a BI, a physical indicator, and a cleaning indicator, and another work history concerning the sterilization processing. Evaluation information and analysis information are provided to each base server and the user terminal, as needed. Note that the evaluation analysis server 16 may be configured to perform an evaluation and analysis every time information is accumulated, and update the base-related information and the like at any time. This can always provide the latest information to the user who requires the information.

The base server 14 manages the management information (the history information can be included) concerning the sterilization workflow for each base. The management information of the base server 14 of each base is accumulated in the evaluation analysis server 16 via the network 17. Furthermore, the base server 14 can acquire necessary information, an evaluation result, and the like by accessing the evaluation analysis server 16. For example, the base server 14 may configured to function as a Web browser to acquire screen information and the like by accessing the evaluation analysis server 16 as a Web server via the Internet. Alternatively, the base server 14 may be formed as a cloud system in which the evaluation analysis server 16 is used as a cloud server. The PC 15 can, for example, add information to the management information held in the base server 14 or modify the management information via the network. The PC 15 and the base server 14 can notify the user of the base system of various kinds of information based on the management information held in the base server 14 or the evaluation analysis server 16. The user is notified of the various kinds of information by an arbitrary method, for example, by displaying notification contents on the display of the PC 15, by generating a sound (an alarm or the like) from a loudspeaker (not shown), or by transmitting mail to a PC (not shown) used by user or a mobile terminal carried by the user. Although there are various methods of notifying the user of the various kinds of information, these methods will collectively be expressed by “the base system sends a notification (to the user)”. Note that in the present invention, the user indicates a person involved in the sterilization workflow such as a worker who actually works in each stage of the sterilization workflow or a supervisor who supervises the worker.

The PC 15 is installed in a place where it is necessary to refer to the management information, add information to the management information, or modify the management information in the sterilization workflow. As shown in FIG. 2A, the PC 15 is connected to the measurement apparatus 1. The measurement apparatus 1 can be connected to the network directly without being connected to the PC 15, and it is possible to display a CI result by providing a display unit in the measurement apparatus 1. Note that instead of the PC 15, another PC (not shown) connected to the PC via the LAN may be connected to the measurement apparatus 1. The measurement apparatus 1 is an apparatus that measures the discoloration region of the CI. Note that in this embodiment, the measurement apparatus 1 is configured to function as a reading apparatus that reads the identification information of the CI in addition to measurement of the discoloration region of the CI. The measurement apparatus 1 is installed in each of a place where it is necessary to measure the CI and a place where it is necessary to read the identification information of the CI, and is connected to the PC 15 or the other PC installed in the same place. Note that the network of each base system is not limited to a closed network in the hospital. For example, the sterilization station may be outside the hospital. In this case, the network connects the hospital and the sterilization station outside the hospital. Alternatively, the network in each base may be a closed network such as a local area network (LAN) that is not connected to another network, or may include a LAN and the Internet. For example, the network can be formed by the Internet and the LAN in the hospital connected to the Internet. In this case, it is possible to form each base system by installing the base server 14 outside the hospital and connecting it to the Internet.

In this embodiment, the sterilization processing apparatus 13 is connected to the network in the base, and the PC 15 and the base server 14 can acquire, from the sterilization processing apparatus 13, information to be added to the management information or information to be used to modify the management information. However, the sterilization processing apparatus 13 need not be connected to the network. In this case, based on the information output from the sterilization processing apparatus 13, the worker operates the PC 15 to, for example, add/modify the management information and the history information. Note that the worker needs to operate the PC 15 to, for example, input/update/display the management information and the like. However, for the sake of descriptive convenience, a description of the operation of the PC 15 will be omitted. “The worker inputs information to the PC 15” will also be expressed as “the worker inputs information to the base system” hereinafter. “The worker inputs information to the PC 15 and the base system records the input information” will also be expressed as “the worker records information in the base system” hereinafter. Furthermore, “information presented to the worker is recorded by being displayed on the display of the PC 15, by being output to a printer connected to the PC 15, or by being output to the hard disk or the like of the PC 15” will also be expressed as “the base system presents/outputs information (to the worker)” hereinafter.

In this embodiment, each set (sterilization target) is assigned with identification information. The identification information can be in, for example, the form of a serial number or barcode printed on a seal. Alternatively, the identification information can be recorded in an IC tag. A medium with identification information is attached to, for example, the exterior surface of the packaging member of the set or the sterilization target of the set. Alternatively, the medium can be attached to a CI or BI enclosed in the set. If the serial number or the like is recorded in the CI or BI, the serial number of the CI or BI enclosed in the set can be used as the identification information of the set. In this embodiment, the serial number of the CI or the BI enclosed in the set is used as the identification information of the set. Note that when the achievement degree of the sterilization processing is verified more accurately, both the CI and BI may be enclosed in the set or externally attached.

Arrangement of Each Apparatus

FIG. 2B shows examples of arrangements of the evaluation analysis server 16 and the base server 14 according to this embodiment. Note that the PC 15 and the user terminals 18 and 19 have the same arrangement as that of the base server 14 and a description thereof will be omitted. Note also that the arrangements to be described below are merely examples, and are not intended to limit the present invention. That is, each apparatus may be formed by including another control component or load.

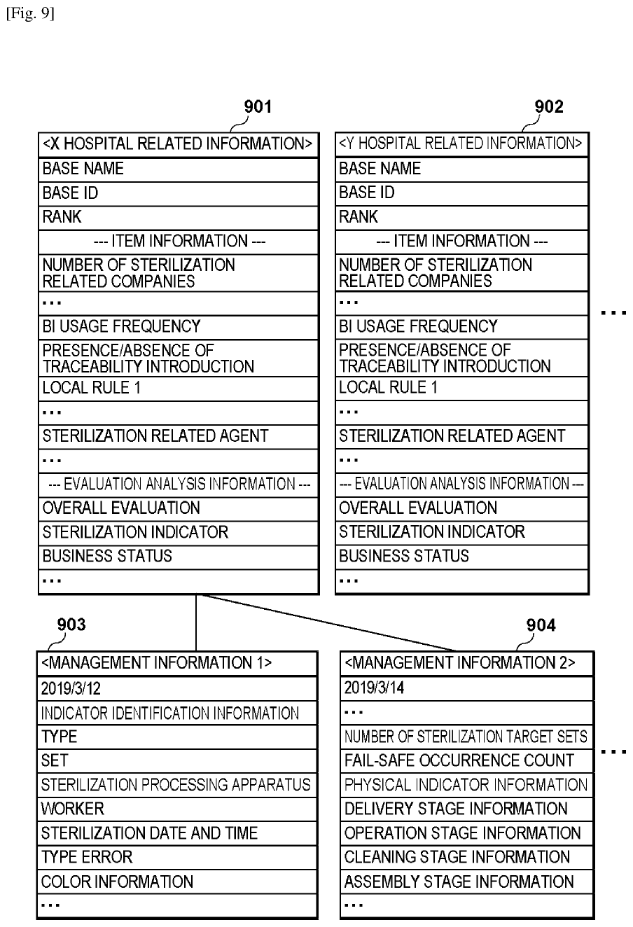

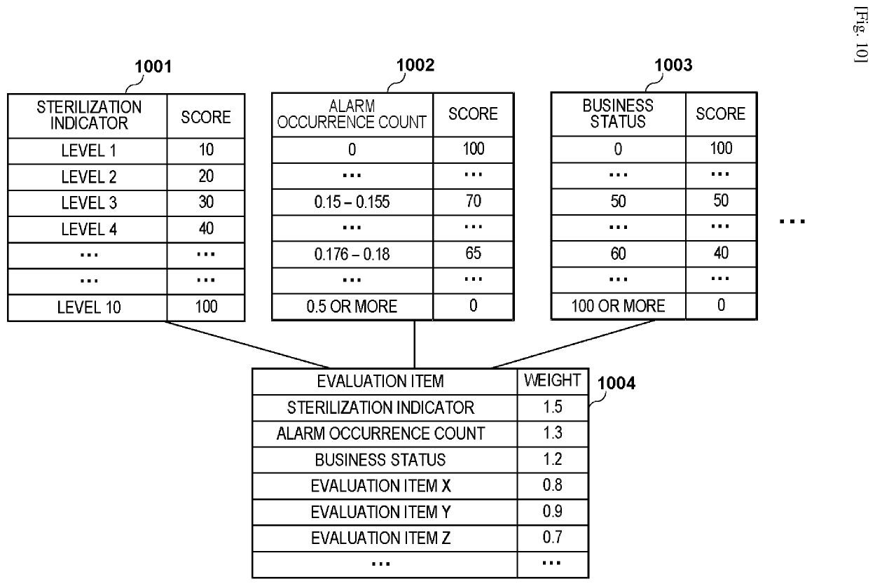

The evaluation analysis server 16 includes a CPU (Central Processing Unit) 201, a RAM (Random Access Memory) 202, a ROM (Read-Only Memory) 203, an HDD (Hard Disk Drive) 204, a database I/F 205, and a network I/F 206. The respective loads are connected via a system bus 210 so as to transmit/receive various signals. The CPU 201 implements various functions by reading out a control program stored in advance in the ROM 203 or the HDD 204 into the RAM 202 and executing it. The ROM 203 stores a boot program, a control program, various setting values, and the like. As described above, the RAM 202 is a work area of the CPU 201, and can temporarily store various kinds of information and parameters. The HDD 204 is a nonvolatile mass storage device, and can store various kinds of programs and information. The database I/F 205 controls data transmission/reception to/from a database 207 communicably connected to the evaluation analysis server 16. The database 207 accumulates, for example, the base-related information for each base, the management information, the history information, and the like. Details of the base-related information will be described later with reference to FIG. 9. The network I/F 206 controls communication with an external apparatus via the network 17. For example, the network I/F 206 can transmit/receive information to/from a network I/F 215 of the base server 14 via the network 17. This makes it possible to acquire various kinds of information from the base server and provide evaluation information and the like, as needed. Similarly, the evaluation analysis server 16 can access the user terminal 18 or 19 via the network 17 to provide information corresponding to the access level of the user. The CPU 201 controls the overall evaluation analysis server 16.

The base server 14 includes a CPU 211, a RAM 212, a ROM 213, an HDD 214, the network I/F 215, an output I/F 216, and an input I/F 217. The respective loads are connected via a system bus 220. A display 218 is connected to the output I/F 216, and an operation unit 219 is connected to the input I/F 217. In the case of the user terminal 18, the touch panel liquid crystal display of a smartphone or the like is connected, and the display 218 and the operation unit 219 are integrally provided. The CPU 211 implements various functions by reading out a control program stored in advance in the ROM 213 or the HDD 214 into the RAM 212 and executing it. The ROM 213 stores a boot program, a control program, various setting values, and the like. As described above, the RAM 212 is a work area of the CPU 211, and can temporarily store various kinds of information and parameters. The HDD 214 is a nonvolatile mass storage device, and can store various kinds of programs and information. The network I/F 215 controls communication with an external apparatus via the network 17. As described above, the network I/F 215 can transmit/receive information to/from the network I/F 206 of the evaluation analysis server 16 via the network 17. The output I/F 216 controls display of the display 218. The input I/F 217 accepts a user operation input via the operation unit 219, and transfers it to the CPU 211.

Functional Components of Each Server

FIG. 2C shows the functional components of each server apparatus according to this embodiment. The functional components of each apparatus to be described below are implemented by the above-described components such as the CPU, the ROM, the RAM, and the like. Note that the functional components to be described below are merely examples, and are not always essential to implementation of the present invention. For example, alternative functional components or additional functional components may be included.

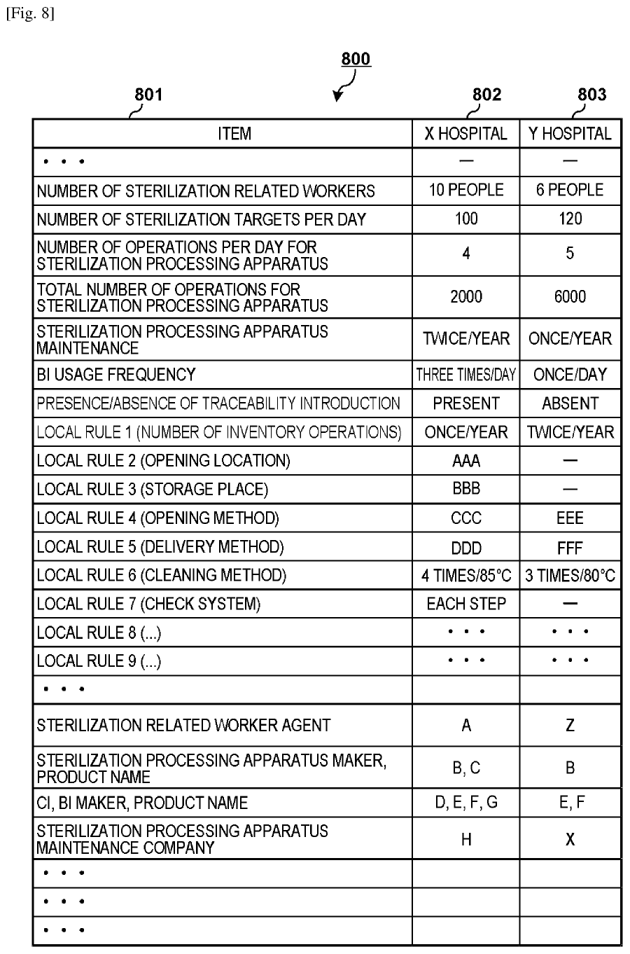

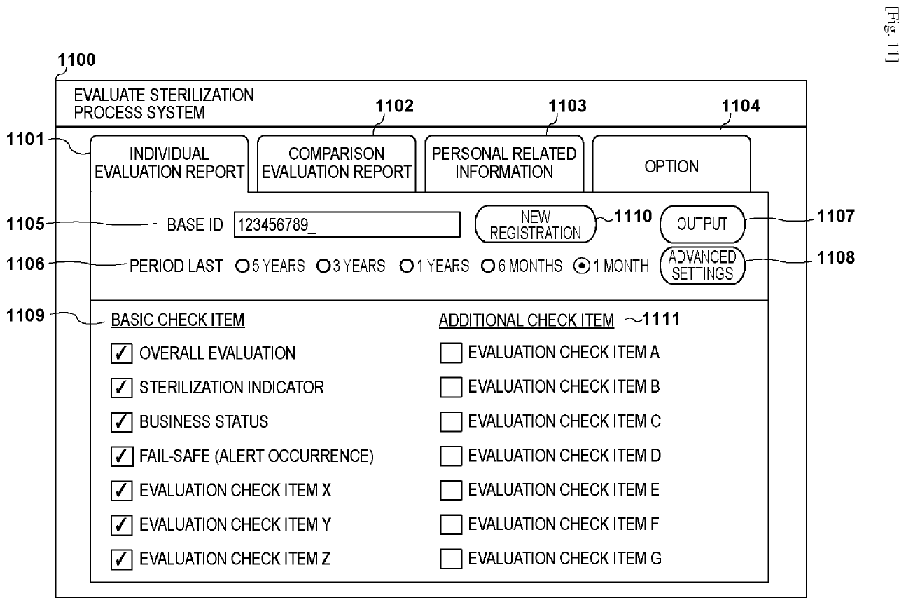

The evaluation analysis server 16 includes, as functional components, an analysis unit 231, a comparison unit 232, a table group 233, a database control unit 234, an evaluation unit 235, and a communication control unit 236. The analysis unit 231 analyzes registration information or various kinds of input information input via the base server 14 or the user terminal 18 or 19. For example, the analysis unit 231 determines, based on the input information, whether information necessary to evaluate an evaluation target item has been obtained. Details of the registration information will be described later with reference to FIG. 8. The input information includes various kinds of information such as information concerning a base, information concerning an evaluation item, information concerning settings of an evaluation system, and information concerning the user. If the accumulation information in the database 207 is updated, the analysis unit 231 evaluates a predetermined evaluation item and calculates the similarity using the evaluation unit 235 and the comparison unit 232, and updates the base-related information for each base accumulated in the database 207. The evaluation unit 235 evaluates the evaluation target item using the information input via the base server 14 or the user terminal 18 or 19, the accumulation information accumulated in the database 207, and the table group 233, and also outputs an individual evaluation report (to be described later). The table group 233 includes, for example, evaluation tables to be described later with reference to FIG. 10. The comparison unit 232 extracts other bases similar to the evaluation target base, acquires evaluations of the plurality of bases extracted by the evaluation unit 235, and outputs a comparison evaluation report (to be described later). The communication control unit 236 controls communication with the base server 14 or the user terminal 18 or 19 via the network 17, and controls data transmission/reception. The database control unit 234 manages data input/output to/from the database 207.

The base server 14 includes, as functional components, a collection unit 241, an alarm unit 242, an input unit 243, a communication control unit 244, a stage management unit 245, and an output unit 246. The collection unit 241 collects pieces of information from various kinds of apparatuses involved in sterilization processing and information input from the user, and manages them as management information and history information (both will be described later). In accordance with the input information, the alarm unit 242 notifies, by generating an alarm or the like, the user of the fact that the achievement degree of the sterilization processing does not satisfy a condition, an error of a medical instrument incorporated in a set in an assembly stage, or the like. The present invention is not limited to this, and the alarm unit 242 may control to display a warning on the display unit of an associated apparatus. The input unit 243 accepts, for example, a user input via the operation unit 219 of the apparatus or an input from another apparatus in the base via the LAN. The output unit 246 also functions as a display control unit, and displays a user interface or the like on the display 218 in accordance with the evaluation information and screen information received from the evaluation analysis server 16. The communication control unit 244 controls communication with the evaluation analysis server 16 or another apparatus in the base via the network 17 or the LAN laid out in the base, and controls data transmission/reception. The stage management unit 245 is a unit that manages each stage associated with the sterilization workflow described with reference to FIG. 1, and issues a work instruction to a worker in accordance with the input information or generates a work history of a worker to notify the collection unit 241 of it.

Chemical Indicator (CI)

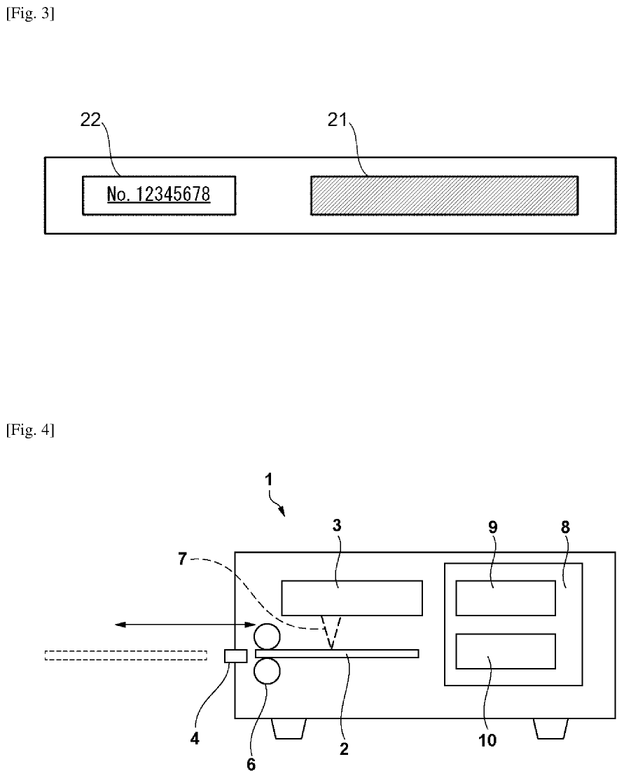

FIG. 3 shows a CI according to this embodiment. The CI is sheet-like test paper, and includes a discoloration region 21 with a surface having undergone chemical processing. The color of the discoloration region 21 changes in accordance with the achievement degree of the sterilization processing. For example, the discoloration region 21 of the CI is configured so that a color value C #1 is set in an initial state, the color value changes to color values C #2 to C #9 in accordance with the achievement degree of the sterilization processing, and the color value is finally constant at a color value C #10. The CI also has an identification portion 22 including identification information. In FIG. 3, the identification information is a serial number. Note that as described above, the identification portion 22 can be a printed barcode or an IC tag (wireless tag) attached to the CI. In this embodiment, assume that the type of the CI can be determined based on the identification information of the identification portion 22. For example, if there are two types of CIs of T1 and T2, the first number of the serial number can indicate the type of the CI.

Measurement Apparatus

FIG. 4 is a sectional view showing an example of the measurement apparatus 1. Note that in FIG. 4, reference numeral 2 denotes a CI conveyed to the measurement position of the measurement apparatus 1, and a dotted line indicates the CI before it is inserted into the measurement apparatus 1. The CI inserted into an insertion portion 4 of the measurement apparatus 1 is conveyed to the measurement position by a conveyance roller pair 6. A measurement unit 3 measures the color of the discoloration region 21 of the CI. After measuring the CI, the CI is discharged outside the measurement apparatus 1 by reversely rotating the conveyance roller pair 6. The measurement unit 3 reads, by light 7, an optical measurement value associated with the color value of the discoloration region 21 of the CI, for example, a spectral reflectance. In this embodiment, the measurement unit 3 is configured to read the identification information of the CI in addition to the discoloration region 21 of the CI. If the identification portion 22 is a serial number or a barcode, the measurement unit 3 can optically read the identification portion 22 of the CI. Note that if the identification portion 22 is an IC tag, the measurement unit 3 includes an IC reader, and reads the identification information stored in the IC tag by communicating with the IC tag. Furthermore, the measurement unit 3 can be configured to read the color value of the discoloration region 21 and the identification information by capturing the discoloration region 21 and the identification portion 22 by an image sensor. A control unit 8 controls the measurement apparatus 1. A storage unit 9 of the control unit 8 stores various kinds of information to be used for measurement. An arithmetic processing unit 10 of the control unit 8 determines the color value of the discoloration region 21 of the CI based on the measurement result of the measurement unit 3, for example, the spectral reflectance. The control unit 8 outputs, to the PC 15, color information indicating the determined color value and the read identification information of the CI.

Note that the measurement apparatus 1 shown in FIG. 4 discharges the CI from the insertion portion 4. However, a discharge portion may be provided on the opposite side of the insertion portion 4 with respect to the measurement position of the CI, and the CI may be discharged from the discharge portion after measurement. Furthermore, the measurement apparatus 1 shown in FIG. 4 measures the CI by the fixed measurement unit 3 after conveying the CI to the measurement position but can be configured so that the measurement unit 3 is movable. That is, the measurement apparatus 1 can be configured to move the measurement unit 3 in a predetermined direction and read the discoloration region 21 and the identification portion 22 of the CI. In addition, the worker can move the measurement apparatus 1 above the CI to read the discoloration region 21 and the identification portion 22 of the CI. Such arrangement is advantageous when CIs of various sizes are used.

Note that if the identification information of the CI is not used as the identification information of the set, identification information is read in accordance with the form of identification information or a medium in which identification information is recorded. For example, if the identification information is a barcode, a barcode reader can be used to read the identification information. Alternatively, if the identification information is a serial number, the worker can input the identification information to the PC 15.

Biological Indicator

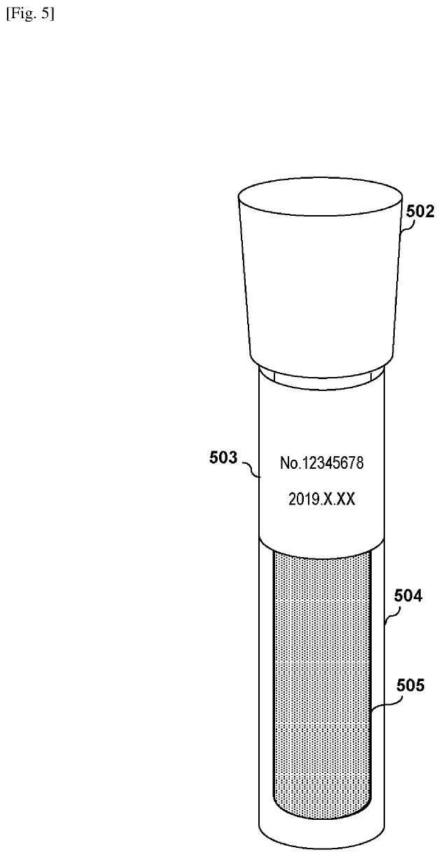

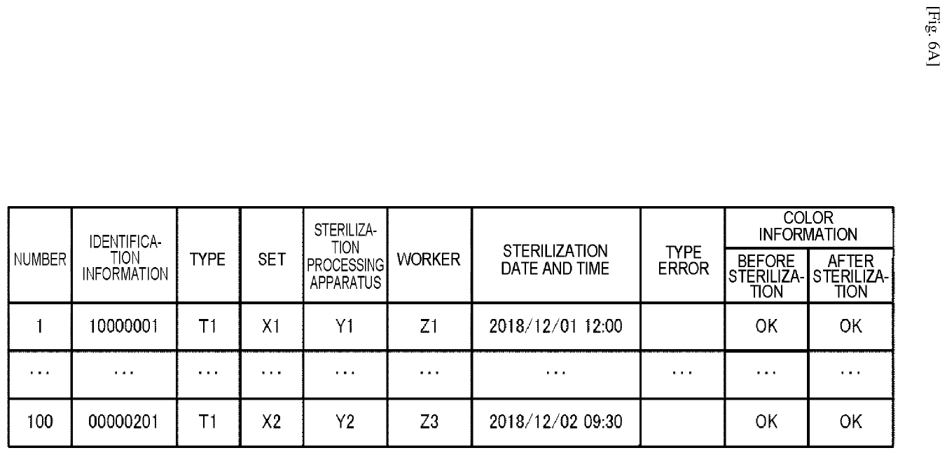

FIG. 5 is a view showing an example of a biological indicator (BI). The BI is an only indicator that can directly verify a microbe-killing effect in the sterilization stage, and can ensure sterility. After the sterilization processing, the BI attached to the set or enclosed in the set is extracted, and cultivated in a procedure recommended by a manufacturer, thereby determining a result. The BI does not react to only a specific important process variable unlike CI, and can directly verify a microbe-killing effect in the sterilization stage. As shown in FIG. 5, the BI is a cylindrical circular structure that holds indicator bacteria in a sealed state. A cover portion 502 is attached to a package container 504, and the package container 504 contains a liquid culture medium 505 including indicator bacteria. Reference numeral 503 denotes a label stuck to the package container 504. On the label, identification information for identifying the BI and information of a use period and the like are printed. Note that this is not intended to limit the printed information. The BI becomes a highly reliable indicator for confirming the sterilization effect by using indicator bacteria suitable to various sterilization methods. In verification using the BI, after sterilization is performed by the sterilization processing apparatus together with the set as the sterilization target, cultivation and determination are performed by a predetermined instrument and the like. Therefore, a time period of about several hours to several days is generally required for verification using the BI. However, the BI may require the cost and processing time, and may be used not every time but periodically (although the period is different for each base, for example, once a day). Thus, it is possible to evaluate the reliability of the sterilization processing system in accordance with the use frequency of the BI.

Physical Indicator

The sterilization processing system according to this embodiment verifies the achievement degree of the sterilization processing and the like using a physical indicator in addition to the above-described CI and BI. The physical indicator corresponds to information indicating the operation history of the apparatus used in the sterilization processing system, for example, the sterilization processing apparatus 13 or the measurement apparatus 1. The information indicating the operation history may include, for example, information of various kinds of sensors, a thermometer, a pressure gauge, a clock, a measurement value, and the like, and information indicating a working history in each apparatus. It is possible to confirm a change amount and an achievement status of an appropriate condition (temperature condition) and the like for each operation by monitoring these pieces of information. It is also possible to monitor these pieces of information in real time by the PC communicably connected to the sterilization processing apparatus, the measurement apparatus, or the like, and to detect an operation abnormality of the apparatus or the like in real time.

Management Information of Base Server

FIGS. 6A-6C show master information tables each of which is one of pieces of management information held in the base server 14 according to this embodiment. The management information to be described here is management information for each set as a sterilization target. Although information concerning the CI will be exemplified as the management information, information concerning various kinds of data of the above-described BI, physical indicator, and cleaning indicator, and the like can be managed together. Furthermore, in the base system according to this embodiment, various kinds of information concerning the sterilization processing system are accumulated as management information and history information without limitation to the management information to be described below. These pieces of information are accumulated in the evaluation analysis server 16 via the network 17. Data to be accumulated are periodically collected for each base. Note that the pieces of information accumulated in the evaluation analysis server 16 will be described later with reference to FIG. 9. A method of inputting and managing some of data finally accumulated in the evaluation analysis server 16 will now be described. Note that the pieces of management information may manually be input by the manager of each base or the like using the base server 14 or the PC 15, or may be pieces of information (information concerning the physical indicator and the like) output from the apparatuses installed in each base, for example, the sterilization processing apparatus 13, the measurement apparatus 1, and the like.

A number field shown in FIGS. 6A-6C indicate the number of each record. Each record is provided for the identification information of each set. An identification information field stores the identification information of the set. In this embodiment, the identification information of the set is the identification information of the CI (or BI) enclosed in the set. A type field stores information indicating the type of the CI specified based on the identification information. A set field stores information for specifying a corresponding set type. A sterilization processing apparatus field stores information for specifying the type of the sterilization processing apparatus 13 that performs sterilization of the set. A worker field stores information for specifying a worker who performs sterilization processing. A sterilization date/time field stores information for specifying a date/time when the sterilization processing is performed. A type error field stores information indicating whether the worker mistakes the type of the CI. A color information field includes two subfields of prior- and post-sterilization subfields. In this embodiment, the measurement apparatus 1 measures the discoloration region 21 of the CI before the CI is used in the sterilization processing, and it is determined based on a measurement result whether the CI has an initial failure. A determination result is stored in the prior-sterilization subfield. In addition, the measurement apparatus 1 measures the discoloration region 21 of the CI of the set having undergone the sterilization processing, and it is determined based on a measurement result whether the sterilization processing has reached a predetermined achievement degree. A determination result is stored in the post-sterilization subfield. The information of each field recorded in each record will also referred to as sterilization-related information of the set hereinafter.

If, for example, contents in the master information table are as shown in FIG. 6A, the packaging stage is executed. At this time, the worker operates the base system to add a new record (number: 101) as a record target record to the master information table. Then, the worker inputs, to the base system, information for specifying the type of the target set of the packaging stage, information for specifying the type of the sterilization processing apparatus to be used, and information for specifying the worker. This records these pieces of information in the set field, the sterilization processing apparatus field, and the worker field of the record target record. At this time, the base system displays, to the worker, the type of a CI to be used, by using a type determination table (to be described later). The worker causes the measurement apparatus 1 to read the CI of the displayed type. In this example, assume that the measurement apparatus 1 reads the CI with identification information “12345678” shown in FIG. 3, and outputs, as a measurement result, a color value (to be referred to as a measured color value hereinafter) Ct of the discoloration region 21 and the identification information “12345678” to the PC 15. After confirming that there is no record corresponding to the identification information “12345678” in the master information table, the PC 15 records the identification information “12345678” in the identification information field of the record target record.

In addition, the PC 15 determines whether the color difference between the color value C #1 of the initial color of the discoloration region 21 of the CI and the measured color value Ct exceeds a threshold. If the color difference between the color value C #1 and the measured color value Ct does not exceed the threshold, the PC 15 records “OK” in the prior-sterilization subfield. Note that if the color difference between the color value C #1 and the measured color value Ct exceeds the threshold, the PC 15 records “NG” in the prior-sterilization subfield. At the same time, the PC 15 displays, on the display unit, information indicating whether the color difference between the color value C #1 and the measured color value Ct exceeds the threshold. If the color difference between the color value C #1 and the measured color value Ct exceeds the threshold, the base system can display, to the user, information indicating that the CI cannot be used in the subsequent stage. Based on the information displayed on the display unit of the PC 15, the worker encloses the measured CI in the set or discards the CI to measure a different CI.

In FIG. 6B shows a state in which the CI enclosed in the set corresponding to the record with the number “101” added as described above has no initial failure and thus the sterilization processing of the set is then performed. In the sterilization date/time field, a data/time when the sterilization processing is performed is recorded. If contents in the master information table are as shown in FIG. 6B, the set including the CI with the identification information “12345678” is used in the surgery stage. In this case, the measurement apparatus 1 measures the CI. This outputs the measured color value Ct and the identification information “12345678” to the PC 15. The PC 15 searches for a record target record corresponding to the identification information “12345678” from the master information table, and confirms that there is only one record target record. Subsequently, the PC 15 determines, among the plurality of color values C #1 to C #10 that can be taken by the discoloration region 21, a color value whose color difference with respect to the measured color value Ct is smallest. If, for example, any one of the color values C #8 to C #10 has a smallest color difference with respect to the measured color value Ct, it is determined that a target achievement degree has been reached (OK); otherwise, it is determined that the target achievement degree has not been reached (NG). If OK is determined, the set is used. If NG is determined, sterilization processing is performed again for the set. The PC 15 records a determination result in the post-sterilization subfield of the record target record while displaying the determination result to the user.

For example, a set corresponding to a record in which data are recorded only in the identification information, type, set, and sterilization processing apparatus fields and the prior-sterilization subfield is a set after the packaging stage and before the sterilization stage. A set corresponding to a record in which data is added to only the sterilization date/time field is a set after the sterilization stage and before the surgery stage (use stage). A record in which data is recorded in the post-sterilization subfield of the color information field corresponds to a set after the surgery stage (use stage). As described above, based on the sterilization-related information, it is possible to determine any of the packaging stage, sterilization stage, and surgery stage, which is complete for the set.

If the worker performs an operation different from a required one in each stage, that is, an erroneous work, the base system notifies the worker of it. For example, if the worker is to enclose, in the packaging stage, a CI of a type which should not be used for the packaging target set, the worker is notified of a CI type error based on the type determination table. FIG. 7 shows the type determination table held in the base server 14. The type determination table (type determination information) shown in FIG. 7 shows the type of a CI to be used for a pair of the type of the sterilization processing apparatus 13 and the type of a set. The type of the sterilization processing apparatus may be decided in advance in accordance with the type of a set, and if the type of the sterilization processing apparatus is decided, the type of a CI may be decided. Referring to FIG. 7, for example, it is defined that if a set of a type X1 undergoes sterilization processing by the sterilization processing apparatus 13 of a type Y1, a CI of a type T1 is used. In addition, referring to FIG. 7, it is defined that if a set of a type X3 undergoes sterilization processing by the sterilization processing apparatus of a type Y3, a CI of a type T2 is used. FIG. 7 also shows that it is impossible to perform sterilization processing for a set of a type X2 by the sterilization processing apparatus of the type Y3.

Assume, for example, that in the packaging stage, the worker records “X1” in the set field and “Y1” in the sterilization processing apparatus field. Based on the type determination table, the base system displays on the display unit that the CI of the type T1 is used. However, if the worker causes the measurement apparatus 1 to erroneously read a CI of the type T2, the base system records “presence” in the type error field, as shown in FIG. 6C. Furthermore, the base system notifies the worker of a type error of T2. As described above, it is possible to prevent the worker from using a CI of an erroneous type by determining, based on the type determination table, the type of a CI to be used.

To maintain the quality of the sterilization workflow, it is necessary to reduce the frequency of an erroneous operation by the worker. For example, in this embodiment, the fact that the measurement apparatus 1 is caused to read a CI of an erroneous type is recorded in the master information table, and it is thus possible to make an improvement based on the record accumulated in the master information table so as to prevent erroneous determination of the type of a CI.

Note that in this embodiment, the error of the type of the CI to be used is used as an example of a work error. The present invention, however, is not limited to this. For example, an operation that does not perform a start operation of the sterilization processing on the sterilization processing apparatus 13 in the sterilization stage, an error of a program used in the sterilization processing apparatus 13, or the like can be detected as a work error and a notification of it can be made. If a work is delayed from a predetermined work time, a delay time may be detected and a notification of it may be made. Information necessary to detect these data is recorded as sterilization-related information. In this embodiment, when determination is performed based on the type determination table, the CI type error is determined by assuming that the data in the set field and sterilization processing apparatus field input by the worker are correct. However, if a combination of the type field, set field, and sterilization processing apparatus field input by the worker does not match a combination shown in the type determination table, the worker may be notified of it.

Note that in this embodiment and the following embodiments, the type of the sterilization processing apparatus 13 is recorded in the sterilization processing apparatus field. However, instead of or in addition to the type of the sterilization processing apparatus 13, information for specifying the individual sterilization processing apparatus 13 can be recorded in the sterilization processing apparatus field.

Basic Registration Information

FIG. 8 shows basic registration information for evaluating and analyzing the sterilization processing system for each base according to this embodiment. Pieces of information (items) to be described below are merely examples, and are not intended to limit the present invention, and other information may be included. These pieces of information may be different for each base. To use the system 100 according to the present invention, it is necessary to register, in the system, basic information for each base. Note that not all of the basic registration information to be described below need to be registered, and information of another similar base or average information can be used.

Reference numeral 800 denotes contents of basic information obtained by accumulating pieces of base information 802 and 803 registered for respective bases. The basic registration information 800 is held in the database 207 of the evaluation analysis server 16. To use the system, the administrator of each base server 14 needs to register base information (to be described below) as information for evaluating and analyzing the sterilization processing system. For the information, it is unnecessary to register all the items, and registration can be performed within a possible range. If it is necessary to use unregistered items and the like for an evaluation and analysis, information set by default or information of a base having high similarity (to be described later) among other bases can be alternatively used. The registered information also includes information that can be updated in accordance with daily accumulation information.

As shown in FIG. 8, items 801 registered as base information include various items associated with the sterilization processing system of each base. For example, the number of involved people for each stage in the sterilization workflow may be registered. The number of sterilization targets per day is an item registered by the number of sets or the number of medical instruments as sterilization targets to undergo sterilization processing per day. As items associated with the sterilization processing apparatus, the number of working per day, the total number of operations, the number of maintenance workings, a maker, a product name, and a maintenance company are included. If there is a sterilization-related agent, information of it can also be registered. Furthermore, as an index indicating whether the safety of the sterilization processing is considered more, items of the use frequency of the BI and the presence/absence of traceability introduction are included. As described above, the BI may change the evaluation of the sterilization processing system in accordance with the use frequency.

The items 801 include items of various local rules. The local rules correspond to information of a method adopted in each base and the like and information indicating a work flow or method in each stage. For example, pieces of information of the number of inventory operations, opening place, storage place, opening method, delivery method, cleaning method, check system, and the like of the sterilization target (set) are included. These local rules are pieces of information which are very useful for evaluating and analyzing the sterilization processing system for each base. Note that if the local rules are known, even if other information is lacking, it is possible to estimate or diagnose the evaluation of the sterilization processing system based on the similarities of the local rules. If, for example, a cleaning temperature and the number of cleaning operations in the cleaning stage are registered as local rules, even if accumulation information such as management information and history information is not held, the evaluation and accumulation information of another base with similar local rules can alternatively be used to perform estimation or diagnosis.

Accumulation Information of Evaluation Analysis Server

FIG. 9 shows the accumulation information accumulated in the evaluation analysis server 16 according to this embodiment. The accumulation information shown in FIG. 9 is information stored in the database 207. Reference numerals 901 and 902 denote pieces of base-related information accumulated for respective bases. The base-related information includes the basic registration information described with reference to FIG. 8 and evaluated and analyzed information, and is information which is updated, as needed, every time management information or the like from each base server is accumulated. The management information acquired from each base is stored in each piece of base-related information in linkage with it. For example, pieces of management information 903 and 904, the basic registration information shown in FIG. 8, and the like are stored in the base-related information 901 of a predetermined base (X hospital) in linkage with it.

The base-related information 901 will be exemplified. The base-related information 901 is information accumulated in the database 207 with respect to “X hospital”. The base-related information 901 includes a base name, a base ID, rank information, item information, and evaluation analysis information. These pieces of information are merely examples, and are not intended to limit the present invention, and other information may be included. The base name and the base ID are the name and identification information of the base associated with the base-related information. The base ID may be unique identification information issued from the system when registration concerning the base is performed, or existing identification information indicating a medical institution. The rank information is information indicating ranking of an evaluation analysis result among the plurality of bases. The item information is information associated with the sterilization processing system of each base including the above-described basic registration information. Information other than the above-described basic registration information may be included. The evaluation analysis information includes information evaluated by the evaluation analysis server 16. As evaluation items, in addition to a plurality of individual evaluation items, a comprehensive evaluation obtained by averaging the evaluation items is included. A comprehensive evaluation acquisition method will be described in detail later. The individual evaluation items include, for example, various items such as a verification result of a sterilization indicator, a business status, and the number of times of occurrence of fail-safe (alarm). An evaluation for each stage may be included.

The pieces of management information 903 and 904 are pieces of information acquired from the base at any time, and are stored for each acquisition date/time. The management information 903 is information corresponding to the management information described above with reference to FIGS. 6A-6C. The management information 904 includes the history information concerning another sterilization processing, different from the management information described above with reference to FIGS. 6A-6C. For example, the number of sterilization target sets per day, the number of times of occurrence of fail-safe, the information of the physical indicator, and information associated with each stage are included. These pieces of management information are uploaded to the evaluation analysis server 16 periodically or every time the information managed by the base server 14 is updated. If the information is accumulated, the evaluation analysis server 16 reacquires the above-described evaluation items, and updates the corresponding base-related information.

Evaluation Method

FIG. 10 shows evaluation tables for respective evaluation items in the evaluation analysis server 16 according to this embodiment. A method of evaluating each evaluation item by the evaluation analysis server 16 will now be described. As shown in FIG. 10, the evaluation analysis server 16 holds evaluation tables 1001, 1002, and 1003 each for scoring (quantifying) each evaluation item in a memory such as the HDD 204. Note that if a capacity is large, these evaluation tables may be stored in the database 207. For the sake of descriptive convenience, the three representative evaluation tables will be exemplified but are not intended to limit the present invention. Evaluation tables concerning other evaluation items are also stored, as a matter of course.

The evaluation table 1001 is an evaluation table for scoring the verification result of a sterilization indicator, particularly, the CI. The verification result of the CI is the measurement result of the discoloration region 21 obtained by color measurement by the measurement apparatus 1. The measurement result is classified into, for example, one of levels 1 to 10, and the respective levels are scored to 10 to 100 points, as defined in the evaluation table 1001.

The evaluation table 1002 is an evaluation table for scoring an alarm (warning) occurrence ratio per day. In this table, a range is provided for an alarm occurrence frequency, and is divided into 100 stages (0 to 1.00 in steps of 0.01), and a score corresponding to each stage is defined. The alarm occurrence ratio basically includes a fail-safe occurrence ratio, and includes an alarm occurrence ratio by an artificial mistake and a failure of an apparatus with respect to an operation amount and work amount per day. As shown in the evaluation table 1002, the occurrence frequency is represented by, for example, 0 to 1.00, and a score is defined for each occurrence frequency.

The evaluation table 1003 is an evaluation table for scoring the business status. In this table, the number of processed sterilization targets per person per day, which is obtained based on the number of workers and the number of sterilization targets (sets) per day, is classified into one of 100 stages, and a score corresponding to each stage is defined. If, for example, the number of processed sterilization targets per person per day is 50, 50 points are given. If the number of processed sterilization targets per person per day is 60, 40 points are given.

The evaluation analysis server 16 holds, in the HDD 204 or the database 207, a weighting table 1004 to be updatable in linkage with the above-described evaluation tables. The weighting table 1004 is a table used to acquire the comprehensive evaluation for each base, and is a table for weighting, in accordance with the degree (importance) of probability of leading to infection, the score of each evaluation item evaluated by each evaluation table. That is, the evaluation analysis server 16 can acquire the comprehensive evaluation for each base by obtaining the weighted average of the scores of the respective evaluation items using the weighting table 1004. Note that the scores of the respective evaluation items may simply be averaged without using the weighting table, thereby acquiring the comprehensive evaluation. The weight for each evaluation item in the weighting table 1004 is merely an example. However, these weights can dynamically be changed in accordance with a daily infection occurrence report and the like. For example, the administrator of the evaluation analysis server 16 may update the weighting table 1004. As described above, it is possible to provide a more reliable evaluation with respect to the sterilization processing system by performing a comprehensive evaluation corresponding to the importance of leading to infection.

Individual Evaluation Report UI

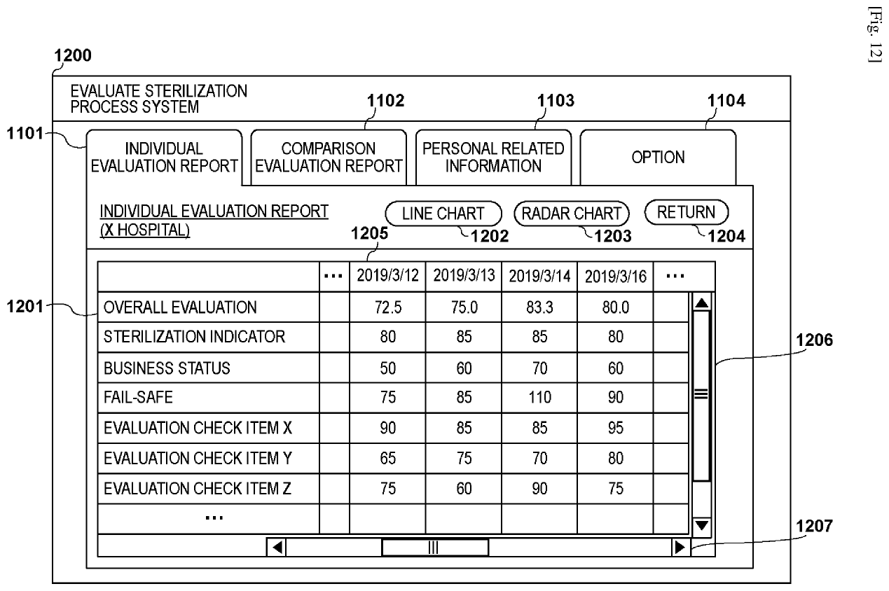

FIG. 11 shows an example of a UI when acquiring an individual evaluation report evaluated for each base by the evaluation analysis server 16 according to this embodiment. A UI 1100 shown in FIG. 11 is basically displayed after the above-described basic registration information is registered, and is displayed on the display unit of the base server 14, the PC 15, or the user terminal 18 or 19 communicably connected via the network 17.

The UI provided by the evaluation analysis server 16 includes tabs 1101 to 1104 for respective provided services, and the UI 1100 shows a state in which the tab 1101 is selected. The tab 1101 provides a UI for outputting the individual evaluation report. Note that the present invention is not limited to this tab arrangement, and this tab arrangement is merely an example. One or more services may be selected in another form. For example, transition may be made from a basic menu screen, in which a plurality of services can be selected, to the UI of each service. The services concerning the remaining tabs will be described in other embodiments.

The UI 1100 is a UI for outputting an evaluation for each base. Note that login authentication to this system may be performed based on the registration information for each base before this screen is displayed, or authentication may be requested when outputting the evaluation. Alternatively, a restriction may be imposed on output contents in accordance with the presence/absence of authentication. The UI 1100 is formed by including an input field 1105 of a base ID for identifying a base (a medical facility or the like) to be evaluated, an input field 1106 of an evaluation period, an output button 1107, an advanced setting button 1108, and a new registration button 1110. As an example, an example of an arrangement capable of designating, in the input field 1105, identification information (name or ID) concerning a predetermined base as an evaluation target of an individual evaluation will be described, and is not intended to limit the present invention, and another evaluation target can be designated. For example, as an evaluation target, at least one of a predetermined sterilization agent, a predetermined sterilization processing apparatus, a predetermined sterilization processing apparatus maintenance company, a predetermined indicator manufacturer, a predetermined outpatient, a predetermined inpatient, and a predetermined surgery patient can be designed. The present invention is not limited to this, and the UI 1100 can be formed by including basic check items 1109 and additional check items 1111 as evaluation items that can be evaluated. When the new registration button 1110 is operated, the screen transitions to a registration screen (not shown) to make it possible to register the basic registration information (FIG. 8) concerning a base to be newly registered. If the base is newly registered, a base ID unique to the target base is issued.

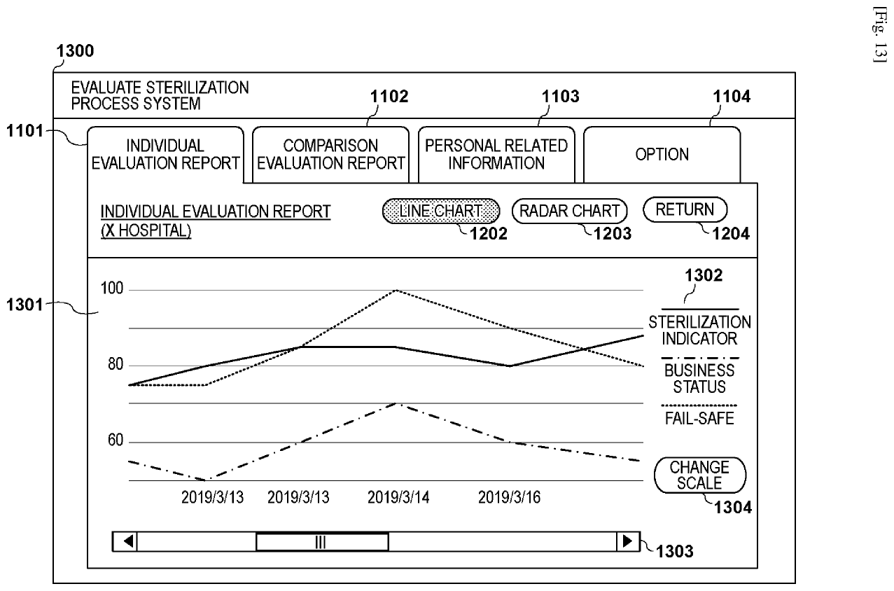

The user can input the base ID to the input field 1105 via the operation unit of each apparatus. Alternatively, a base name may be input to the input field 1105. In the input field 1106, a plurality of radio buttons corresponding to respective periods used for selecting an evaluation period are selectably displayed. If, for example, “1 month” is selected, evaluation output is performed based on management information for the last one month. When the advanced setting button 1108 is operated, the screen transitions to a setting screen (not shown) for making various advanced settings. In this setting screen, for example, the evaluation period can specifically be designated, and a default output format (display format) or default evaluation items as output targets can be designated. As shown in FIG. 11, as for the basic check items 1109, check buttons corresponding to the respective evaluation items are selectably displayed, and are displayed by default in a state in which all the items are selected (checked). Furthermore, every time the check button is operated, the setting state is alternately changed between the selected state and unselected state. As shown in FIG. 11, as for the additional check items 1111 as well, check buttons corresponding to the respective evaluation items are selectably displayed, and are displayed by default in a state in which all the items are not selected. When the output button 1107 is operated in a state in which various settings are made via the UI 1100, a result screen shown in each of FIGS. 12 to 14B is displayed. Note that if an evaluation cannot be executed, for example, if a compulsory selection item is not selected, a popup screen or the like may be displayed to prompt the user to register necessary information or select setting information.

Note that even if the management information for each base is not accumulated, if the basic registration information is registered, it is possible to make an evaluation using data of another base having a similar predetermined item of the basic registration information. The similarity between the bases will be described in the second embodiment. If data of another base cannot alternatively be used, a restriction is imposed on the evaluation items. For example, a restriction is imposed not to select predetermined ones of the basic check items 1109 and the additional check items 1111 shown in FIG. 11, or the items themselves may not be displayed.

Output Example of Individual Evaluation

FIGS. 12 to 14B each show an output example (first user interface) of the individual evaluation report according to the embodiment. As shown in each of UIs 1200 to 1410, an evaluation result evaluated by the evaluation analysis server 16 is visualized and displayed on the user terminal (base server 14, PC 15, or user terminal 18 or 19) used by the user. The UI 1200 shown in FIG. 12 is displayed when the output button 1107 is operated in the UI 1100. Note that the other UI 1300 or 1400 may be displayed first. The UI 1200 can be formed by including, for example, an evaluation result 1201, a line chart button 1202, a radar chart button 1203, and a return button 1204. In the evaluation result 1201, for each predetermined evaluation period 1205, for example, for each day in the example shown in FIG. 12, the scores of the respective evaluation items and the score of the comprehensive evaluation obtained by calculating the average (or weighted average) of the scores are displayed in a table format. By operating (sliding) scroll bars 1206 and 1207, evaluations of other dates and other evaluation items, which are not displayed in the display region of the evaluation result 1201, are displayed.

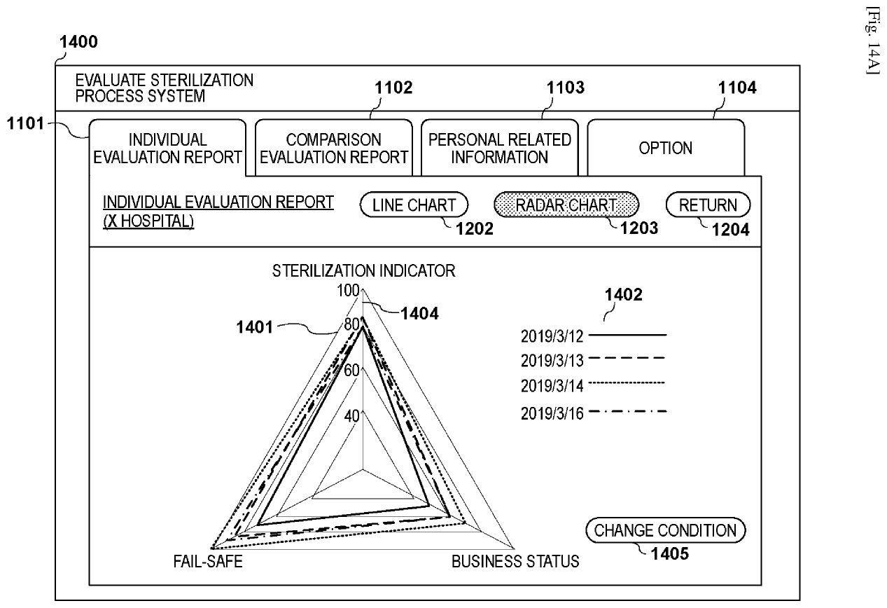

If the line chart button 1202 is selected in the UI 1200, the screen transitions to the UI 1300 shown in FIG. 13. The UI 1300 can be formed by including, for example, an evaluation result 1301, legends 1302, a scroll bar 1303, and a scale change button 1304 in addition to the same various buttons as those in the UI 1200. In the evaluation result 1301, the abscissa represents the date/time indicating the evaluation period, the ordinate represents the score of each evaluation item, and the evaluation results are displayed in time series. The legends 1302 indicate line styles corresponding to the displayed evaluation items. Although an example of outputting three evaluation items is shown, more evaluation items may simultaneously be output. The number of evaluation items to be output can be set in, for example, the setting screen to which the screen transitions when the advanced setting button 1108 of the UI 1100 is operated. Furthermore, the output evaluation items may include the comprehensive evaluation. When the scale change button 1304 is operated, the evaluation period can be changed. If, for example, scores per month are output by changing the evaluation period, output is performed using the average value of the scores within the period of one month. Note that this scale change operation is also applicable to a table format or radar chart as another display format. It is possible to readily confirm evaluations in time series by outputting the evaluation results in a line chart in this way.

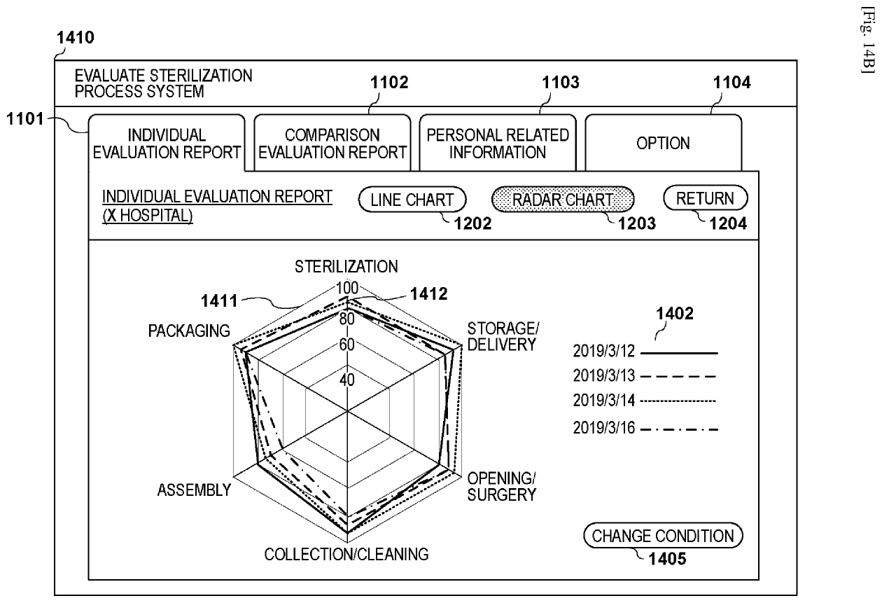

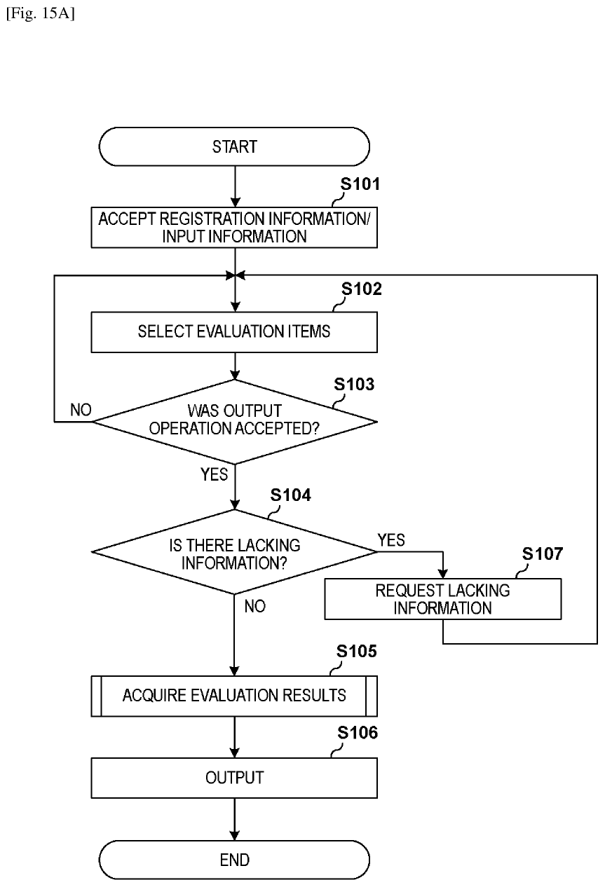

If the radar chart button 1203 is selected in the UI 1200 or 1300, the screen transitions to the UI 1400 shown in FIG. 14A. In this case, for example, the radar chart button 1203 is desirably pressed in a state in which the plurality of evaluation periods 1205 are selected in the evaluation result 1201 of the UI 1200. This is because if the evaluation results of all the periods are to be displayed in the radar chart, a plurality of results are superimposed and displayed, and a display result is thus difficult to read. Therefore, if output is performed in the radar chart, it is desirable to set the upper limit (for example, six) of the number of selectable evaluation periods. Note that control may be executed to select the evaluation periods by selecting date portions. The UI 1400 can be formed by including, for example, an evaluation result 1401, legends 1402, and a condition change button 1405 in addition to the same various buttons as those in the UI 1200. Reference numeral 1404 denotes axes of the radar chart. In the evaluation result 1401, an evaluation for each date of the four evaluation periods (four days) is output in the radar chart. Although an example of outputting three evaluation items is shown, more evaluation items may simultaneously be output. The number of evaluation items to be output can be set in, for example, the setting screen to which the screen transitions when the advanced setting button 1108 of the UI 1100 is operated. It is possible to readily confirm the overall picture of the evaluation result by outputting the evaluation result in the radar chart in this way.

If the condition change button 1405 is operated, it is possible to change the dates to be output and evaluation items to be output. Alternatively, it is possible to change an output condition to a stage-specific evaluation result by operating the condition change button 1405. FIG. 14B shows an example of a stage-specific evaluation result. In the UI 1410, a stage-specific evaluation result 1411 in the sterilization workflow is displayed, instead of the evaluation result 1401 of the UI 1400. Reference numeral 1412 denotes axes of a radar chart. In the evaluation result 1411, evaluation results are displayed by dividing the stages included in the sterilization workflow into, for example, six stages of a sterilization stage, a storage and delivery stage, an opening and surgery stage (use stage), a collection and cleaning stage, an assembly stage, and a packaging stage. When output is changed from the evaluation item-specific evaluation result 1401 to the stage-specific evaluation result 1411, for example, the evaluation value of each stage is acquired by classifying the evaluation values of the evaluation items into one or more stages with which the evaluation items are associated, and averaging the evaluation values of the evaluation items which are associated with each stage. For example, as for the assembly stage, the business status, fail-safe, and evaluation check item Y among the basic check items 1109 shown in FIG. 11 are associated with the assembly stage, and the evaluation of the assembly stage can be acquired based on the results of the three evaluation items. In this case, a simple average value may be obtained or a weighted average corresponding to the degree of association with the stage may be obtained. By outputting an evaluation result for each stage as described above, the user can conform the quality of the corresponding base for each stage, and can readily examine an improvement. In the example shown in FIG. 14B, the user can determine that the evaluation of the assembly stage is low as a whole, and can focus on examining an improvement of the assembly stage, thereby supporting an improvement in quality of the sterilization processing system. Note that output of the stage-specific evaluation result by the radar chart has been exemplified but is not intended to limit the present invention. Output may be performed in any format as long as a chart highlights the evaluation for each stage as the evaluation is lower.

If a button of another output format is operated while each of the above-described UIs 1200 to 1410 is displayed, the screen transitions to a UI screen of the operated output format. If the return button 1204 is pressed, control can be executed to transition to the screen before transition or to the UI 1100 or 1200. The transition destination may be settable in the above-described setting screen of the advanced settings. The formats of the table, line chart, and radar chart have been described as the output formats of the individual evaluation report but are not intended to limit the present invention, and another output format may be applied. Although the output method by display on the display unit has been explained, another output method may be adopted. For example, print output or output by data transmission using email transmission may be performed.

Processing Procedure

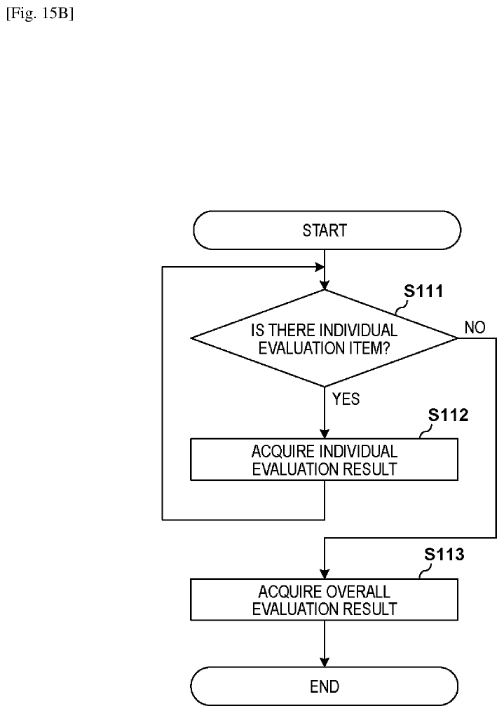

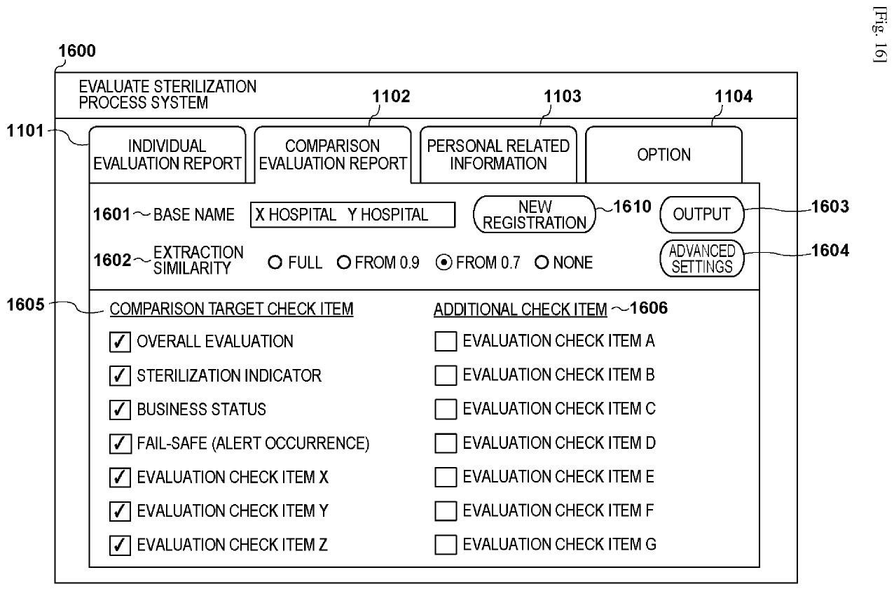

FIG. 15A is a flowchart illustrating a processing procedure when outputting the individual evaluation report according to the embodiment. As a use case of outputting the individual evaluation report, a case in which the user of a predetermined base accesses the evaluation analysis server 16 via the base server 14a will be described. In this case, the UI 1100 in a state in which neither the basic check items 1109 nor the additional check items 1111 are displayed is displayed on the display 218, and is operated by the user. The reason why neither the basic check items 1109 nor the additional check items 1111 are displayed is that the evaluation target base is not selected. Processing to be described below is implemented when, for example, the CPU 201 of the evaluation analysis server 16 reads out a control program stored in the ROM 203 or the HDD 204 into the RAM 202 and executes it. Note that this processing may be executed by each base server 14. In this case, processing of acquiring necessary information from the evaluation analysis server 16 is added. A number following “S” to be described below indicates a step number in the flowchart.

In S101, the CPU 201 accepts registration information or input information of an evaluation target via the network 17. If, for example, base-related information concerning the evaluation target base is not registered, the user registers the basic registration information described with reference to FIG. 8 by operating the new registration button 1110 of the UI 1100. When the new registration button 1110 is operated, the evaluation analysis server 16 notifies the base server 14a of screen information corresponding to a registration screen (not shown), and accepts input by the user. If the basic registration information is already held as base-related information in the database 207 or new registration is complete, the base ID indicating the evaluation target, advanced settings, and the like are accepted as input information. Note that authentication processing of the user may be performed at the timing of starting the processing in S101, or authentication processing may be executed at the timing of operating the output button 1107. That is, the authentication processing is performed before an evaluation result is output, and it is determined whether the user has an access level at which the evaluation result can be accessed. This is done because the evaluation analysis server 16 according to the present invention is configured to release the information not only to an individual medical institution but also to other medical institutions and general users (for example, patients). That is, this is done to prevent managed accumulation information from being used illegally by imposing a restriction on accessible information for each user. After predetermined information is accepted, the process advances to S102.

Upon accepting the input of the evaluation target base, in S102 the CPU 201 notifies the base server 14a of the screen information of the basic check items 1109 and the additional check items 1111 corresponding to the evaluation target base to update the display of the UI 1100. Thus, the CPU 201 accepts selection of evaluation items via the UI 1100. The evaluation items are selected by the checkboxes of the basic check items 1109 and the additional check items 1111. Subsequently, in S103, the CPU 201 determines whether an output operation has been accepted by operating the output button 1107. If the output operation has been accepted, the process advances to S104; otherwise, the process returns to S102.

To acquire evaluations of the accepted evaluation items, the CPU 201 determines in S104 whether there is lacking information. If there is lacking information, the process advances to S107; otherwise, the process advances to S105. Even if there is lacking information, if the basic registration information concerning another similar base can alternatively be used, the process desirably advances to S105 using the alternative information. Alternatively, the user may be inquired that the alternative information from the similar base is used for the lacking information. This allows the user to select to input information supplementing the lacking information if the user has it or to use the alternative information if the user has no supplemental information or information is uncertain. Note that whether the alternative information can be used depends on the specifications and environment of the system, and a detailed description thereof will be omitted. If there is lacking information, in S107 the CPU 201 notifies, via the network 17, the base server 14a of screen information for requesting the lacking information, and acquires the lacking information via the screen, and the process returns to S102. If the lacking information cannot be acquired in S107, selection of the evaluation items is accepted again in a state in which a restriction is imposed not to select the evaluation item that uses the lacking information. If the lacking information can be acquired, the process may advance to S103 by skipping the processing in S102.

On the other hand, if it is determined in S104 that there is no lacking information, the CPU 201 acquires, in S105, evaluations of individual evaluation items as the selected individual evaluation items and a comprehensive evaluation, and outputs the evaluation result in S106, thereby ending the processing. Note that acquisition is a concept including acquisition of an evaluation value (score) using the above-described evaluation table, and calculation of an evaluation value using a predetermined formula or the like. In cases to be described below as well, the concept is included unless otherwise specified. The evaluation result can be output in the format of each of the above-described UIs 1200 to 1410 or another format. Note that an output format suitable to the selected evaluation items may automatically be selected.

FIG. 15B is a flowchart illustrating the subroutine (S105) of the individual evaluation processing according to the embodiment. In S111, the CPU 201 determines whether there is an individual evaluation item to be evaluated. If there is an individual evaluation item, the process advances to S112; otherwise, the process advances to S113. In S112, the CPU 201 acquires a score as an evaluation value in accordance with the input information associated with the evaluation item with reference to the evaluation table corresponding to the target evaluation item. The input information is, for example, level information of an evaluation result for the evaluation table 1001 shown in FIG. 10. The information may be acquired by user input in S101 or S107 described above, or can also be acquired from the management information of the evaluation target base already accumulated in the database 207. As described above, in S112, an evaluation value is acquired using the corresponding evaluation table in accordance with the evaluation item. Note that if there is no corresponding evaluation table, an evaluation value may be acquired using a predetermined formula or the like. On the other hand, if it is determined in S111 that there is no individual evaluation item, in S113 the CPU 201 obtains a weighted average using the acquired evaluation values (scores) of the individual evaluation items and the weighting table 1004 to acquire the evaluation value of the comprehensive evaluation, and returns the process to S105 as the main routine. Although the example of using the weighted average has been explained, the average value of the acquired scores of the respective evaluation items may simply be used as the comprehensive evaluation.

As described above, the system according to this embodiment is a system that evaluates the equality of the sterilization workflow concerning sterilization of a sterilization target. This system registers, for each base, the basic information concerning the base where the sterilization workflow is performed, acquires, for each base, accumulation information indicating the results of works associated with the sterilization workflow, and accumulates the information. In accordance with the evaluation target and the evaluation item designated by the user of the system, the system evaluates at least one evaluation item associated with at least one stage of the sterilization workflow using the accumulation information and the basic information concerning the evaluation target. Furthermore, this system visualizes and provides the evaluation result to the user terminal. According to this embodiment, it is possible to preferably perform a quality evaluation in the sterilization processing system, and preferably provide the quality evaluation and related parameters, as needed. In addition, the evaluation result can be output for a supplying department or surgery department as an associated work department, for a hospital's work or sterilization agent's work if there is a sterilization agent, for an outpatient/inpatient/surgery patient, or for each clinical department. Although the above-described embodiment has exemplified the base as an evaluation target, the evaluation target can be changed to a sterilization agent, an apparatus or indicator manufacturer, an outpatient/inpatient/surgery patient, a clinical department, or the like. In this case, information of each evaluation target is desirably registered as the basic registration information instead of the base information.

### Second Embodiment

The second embodiment of the present invention will be described below. The above first embodiment has explained the example of providing the individual evaluation report associated with a predetermined base of the sterilization processing system. However, this embodiment will describe, for example, provision of a comparison evaluation report in which the evaluation results of a plurality of bases can be compared with each other. A description will be provided by exemplifying bases as comparison targets. However, the comparison targets are not limited to the bases, as will be described in the following modification, and the present invention is applicable to other comparison targets. Details will be described later. Note that the same reference numerals as in the above embodiment denote similar components and controls, and a description thereof will be omitted. This embodiment will describe a function corresponding to the tab 1102, and is applicable in combination of the above-described first embodiment, as a matter of course.

Comparison Evaluation Report UI

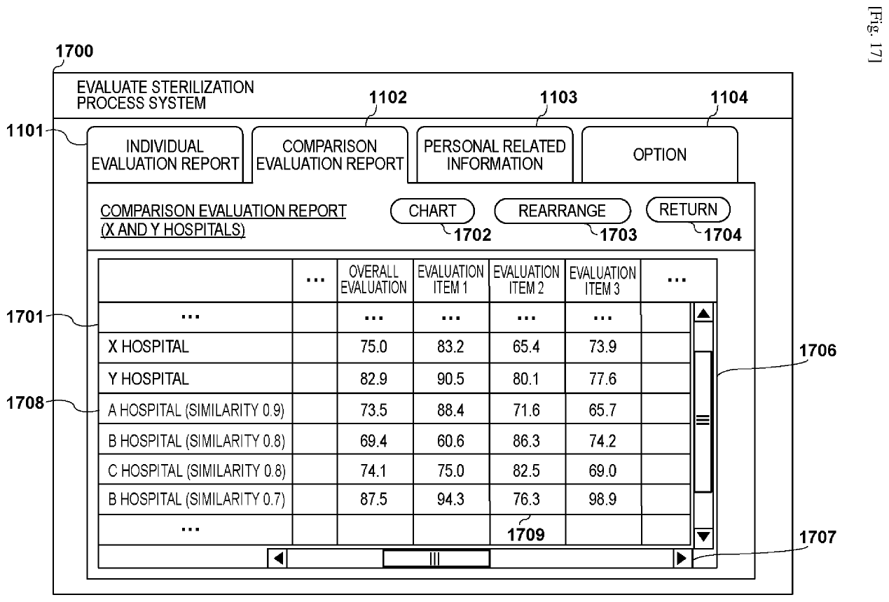

FIG. 16 shows an example of a UI when acquiring a comparison evaluation report by performing evaluation over a plurality of bases by an evaluation analysis server 16 according to this embodiment. A UI 1600 shown in FIG. 16 is displayed on the display unit of a base server 14, a PC 15, or a user terminal 18 or 19 communicably connected to the evaluation analysis server 16 via a network 17. Authentication of the user may be performed before or after the UI 1600 is displayed on the display unit of each apparatus. However, to perform comparison evaluation, comparable items are decided in accordance with the access level of the user. It is thus necessary to perform authentication of the user before comparison target evaluation check items to be described below are displayed.

In the UI 1600, among tabs 1101 to 1104 described above with reference to FIG. 11, the tab 1102 is selected. The tab 1102 is a user interface that provides evaluations of sterilization processing systems in a plurality of bases so as to compare the evaluations. Note that at the stage in which the UI 1600 shown in FIG. 16 is displayed, authentication of the user has already been performed. The UI 1600 can be formed by including at least input fields 1601 and 1602, an output button 1603, an advanced setting button 1604, a new registration button 1610, comparison target check items 1605, and additional check items 1606. The input field 1601 is a field to which a base name or a base ID is input as comparison target identification information. The input field 1602 is an input field to which similarity between bases (comparison targets) extracted as comparison targets is set. The output button 1603 is a button used to output, using set contents, a comparison evaluation report as a comparison result. The advanced setting button 1604 is a button used to transition to a screen for making various settings concerning output of the comparison evaluation report, and makes it possible to specifically set, for example, an item when detecting the similarity in addition to contents of an advanced setting button 1108 described above. As the item when detecting the similarity, it is possible to set only items associated with local rules, and thus possible to compare the evaluation of the sterilization processing system with that of another base with similar local rules. In the default settings, all of the above basic registration information is targeted. The new registration button 1610 is a button having a function similar to that of the new registration button 1110, and a description thereof will be omitted.

One or more base names or base IDs can be input to the input field 1601. The similarly when extracting another similar base from information accumulated in a database 207 can be set in the input field 1602. If, for example, comparison target bases are already narrowed, the user inputs the base names or base IDs of the bases to the input field 1601, and sets extraction similarity of another similar base of the input field 1602 to “none”. On the other hand, if the user wants to compare the evaluation of the base input to the input field 1601 with that of another similar base, the user sets an item other than “none”, for example, similarity of “0.7” or the like in the input field 1602. The similarity is a value indicating the degree of similarity in one or more items of the basic registration information between the bases, and is a value indicating the similarity in the number of workers, the number of sterilization targets per day, the use frequency of a BI, and the like. To calculate the similarity, a difference in each item between the comparison target bases is classified into one of stages (for example, 100 stages of 0 to 1.00) and calculated, and the average value of the similarities of the comparable items is acquired as the similarity between the bases. Assume that as the value is closer to 1.00, the similarity is higher. Therefore, with respect to the comparison evaluation target base, the basic registration information needs to have been registered in the database 207. Similar to the above-described first embodiment, registration information may newly be registered to perform comparison, as a matter of course.

The comparison target check items 1605 and the additional check items 1606 are selectably displayed when the comparison targets are input. Display of an incomparable item is restricted so as not to be selected. When the output button 1603 is operated, a comparison evaluation result (to be described later) is output.

Output Example of Comparison Evaluation

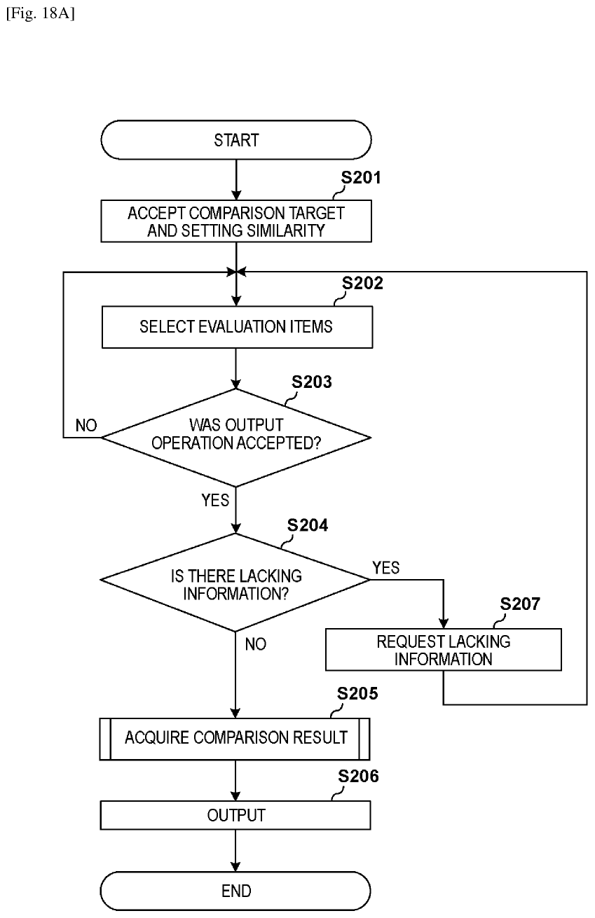

FIG. 17 shows an output example (second user interface) of the comparison evaluation report according to the embodiment. As shown in a UI 1700, evaluation results evaluated by the evaluation analysis server 16 are visualized and displayed on the display unit of the user terminal (base server 14, PC 15, or user terminal 18 or 19) used by the user so as to compare the evaluation results. The UI 1700 can be formed by including a comparison result 1701, a chart button 1702, a sort button 1703, and a return button 1704. In the comparison result 1701, scroll bars 1706 and 1707 for displaying an undisplayed portion are displayed, as needed. The comparison result 1701 is output in a table format but may be displayed in a line chart, a radar chart, or another format, similar to the above-described first embodiment. When changing display of the comparison result to another format, the user can operate the chart button 1702 to select one of the plurality of format options described above.

If the output button 1603 is operated in the setting status of the UI 1600 shown in FIG. 16, for example, the base input to the input field 1601 and comparison results of hospitals with similarities of 0.7 or more are displayed in the comparison result 1701. Furthermore, if the plurality of base names are input to the input field 1601, hospitals with similarities of 0.7 or more with respect to the base (X hospital in FIG. 16) input to the left end of the input field 1601 may be extracted as comparison targets. With respect to each selected evaluation item in the comparison result 1701, if necessary information, for example, management information or the like is not accumulated, control is executed not to display an evaluation value in a predetermined evaluation item of the corresponding base.

When the sort button 1703 is operated, predetermined conditions, for example, the order of the comprehensive evaluation and the order of evaluation item 1 are presented as options, and contents of the displayed comparison result can be sorted. To limit sort targets, a plurality of base names may be selected. If, for example, the user wants to compare X hospital with another hospital in the state shown in FIG. 17, when the cells of the base names other than X hospital are selected and the sort button 1703 is operated, the hospitals other than X hospital are sorted under the selected predetermined condition, for example, in the order of the comprehensive evaluation. In this case, if the comparison targets are only the bases displayed in FIG. 17, the hospitals are sorted in order of X hospital, D hospital, Y hospital, C hospital, A hospital, and B hospital from above. Therefore, the user can readily confirm, among other bases similar to X hospital, a hospital with a high comprehensive evaluation. When the return button 1704 is operated, the screen returns to the screen of the UI 1600.

When each cell in table output is selected in the UI 1700, corresponding detailed information (related parameters) may be displayed. For example, if a cell 1708 of A hospital (similarity of 0.9) is selected, detailed information about A hospital, for example, base-related information (basic registration information) including local rules may be displayed in a format of a popup screen or the like. If a cell 1709 is selected, information used to obtain the evaluation result of evaluation item 2 of D hospital, for example, management information may be displayed as related parameters. If a cell of an evaluation item, detailed information of the evaluation item may be displayed. This allows the user to readily obtain desired information, and readily obtain information useful in improvement of the system. If, for example, another similar base has a higher evaluation, it is possible to readily obtain adopted local rules, and thus readily extract an improvement in the system of the user. If a difference in evaluation is large, highlighting such as color coding can be performed to provide clear presentation. Note that the related parameters can be displayed in the same manner even in the arrangement of the first embodiment described above.

Processing Procedure

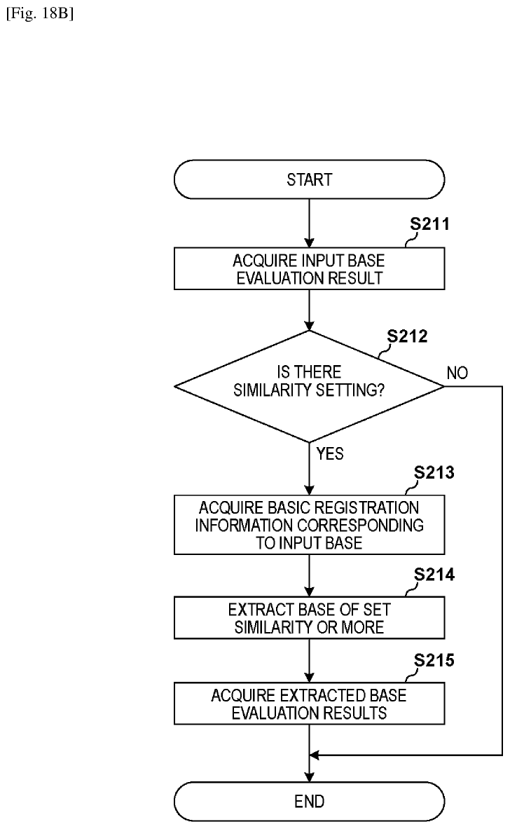

FIG. 18A is a flowchart illustrating a processing procedure when outputting the comparison evaluation report according to the embodiment. As a use case of outputting the comparison evaluation report, a case in which the user of a predetermined base accesses the evaluation analysis server 16 via a base server 14a. In this case, the UI 1600 in a state in which neither the comparison target check items 1605 nor the additional check items 1606 are displayed is displayed on the display 218, and is operated by the user. The reason why neither the comparison target check items 1605 nor the additional check items 1606 are displayed is that the comparison target base is not selected. Processing to be described below is implemented when, for example, a CPU 201 of the evaluation analysis server 16 reads out a control program stored in a ROM 203 or an HDD 204 into a RAM 202 and executes it. Note that this processing may be executed by each base server 14. In this case, processing of acquiring necessary information from the evaluation analysis server 16 is added. A number following “S” to be described below indicates a step number in the flowchart.

In S201, the CPU 201 accepts, via the network 17, the comparison target base information and the similarity setting information input via the UI 1600. S202 to S204 and S207 as processes after that are similar to those in S102 to S104 and S107 of FIG. 15A and a description thereof will be omitted.

If it is determined in S204 that there is no lacking information, the CPU 201 acquires, in S205, a comparison result based on the selected comparison target and similarity, and outputs the acquired comparison result in S206, thereby ending the processing. The comparison result can be output in the format of the UI 1700 described above or another format. Note that an output format suitable to the selected evaluation items may automatically be selected. The comparison result acquisition processing in S205 will be described in detail with reference to FIG. 18B.

FIG. 18B is a flowchart illustrating the subroutine (S205) of the comparison evaluation processing according to the embodiment. In S211, the CPU 201 acquires the evaluation result of the base input to the input field 1601. If a plurality of bases are input, the evaluation result of each base is acquired. An evaluation result acquisition method is similar to that in S112 of FIG. 15B already described above and a detailed description thereof will be omitted. In S212, the CPU 201 determines whether the similarity has been set. If “none” is set in the input field 1602, it is determined that the similarity has not been set; otherwise, it is determined that the similarity has been set. If the similarity has been set, the process advances to S213; otherwise, the process ends and returns to S205.

In S213, the CPU 201 acquires, from the database 207, the base-related information of the base (for example, X hospital) input to the input field 1601. In S214, the CPU 201 extracts a base corresponding to the set similarity information (for example, “0.7” or the like). More specifically, the CPU 201 compares each item of the base-related information acquired in S213 with the base-related information of another base accumulated in the database 207, thereby extracting a base corresponding to the set similarity information. Note that the CPU 201 may calculate the similarity with reference to each piece of information, or may calculate the similarity between the bases in advance and hold a table in which the similarity between the bases is defined. This can reduce similarity calculation processing when a request of the comparison evaluation report is accepted, thereby reducing the processing load. Note that in this case, the CPU 201 acquires base information indicating a value equal to or higher than the set similarity with reference to the table. In S215, the CPU 201 acquires the evaluation results of all the extracted bases, and returns the process to S205. An evaluation result acquisition method is similar to that in S112 of FIG. 15B already described above and a detailed description thereof will be omitted.

As described above, when the user of the system designates a plurality of bases, the system according to this embodiment provides the evaluation results of the plurality of bases so as to compare the evaluation results. If the user of the system designates a predetermined base and similarity, the system provides the evaluation result of the predetermined base and the evaluation result of another base with the degree of similarity with respect to the predetermined base so as to compare the evaluation results, which exceeds the designated similarity. In addition to the various effects of the above-described first embodiment, quality evaluations in a plurality of sterilization processing systems and related parameters can be provided so as to compare the quality evaluations. This allows the user to readily examine an improvement of the sterilization processing system used by himself/herself.

Modifications

The present invention is not limited to the above-described embodiment and various modifications can be made. The above-described embodiment has explained the example of setting bases as comparison targets. However, the present invention is not intended to limit the comparison targets to the bases. For example, in addition to comparison between the base and each of other bases, comparison can be performed between sterilization agents if there are sterilization agents, between apparatus or indicator manufacturers, between related companies such as maintenance companies, between related persons such as outpatients, inpatients, or surgery patients, or between divisions (including regions for a wide area) such as a clinical department. If comparison is performed between sterilization agents, comparison evaluation is performed for evaluation items concerning stages associated with the sterilization agents among the stages of the sterilization workflow, for example, the cleaning stage, assembly stage, packaging stage, sterilization stage, and storage stage. Therefore, the evaluation analysis server 16 extracts, from the base-related information in the database 207, pieces of information associated with a plurality of comparison target sterilization agents, and performs comparison. Note that to reduce the processing load at the time of comparison, related information (agent-related information) for each sterilization agent may be extracted in advance and held in the database 207. Similarly, as for other comparison targets, control is executed to extract related information from the database 207, and perform comparison of related evaluation items. In the above-described embodiment, in the comparison evaluation UI, a base name is input as identification information serving as a comparison target. However, if comparison is performed with respect to another comparison target described above, a change is made to input, as identification information, the name of the other comparison target or the like.

### Third Embodiment

The third embodiment of the present invention will be described below. In each of the above-described first and second embodiments, if the user is engaged in a medical institution, for example, access via the base server of each base has been explained. However, this embodiment will describe information provision to a general user such as a patient. Note that the same reference numerals as in the above-described embodiments denote similar components and controls and a description thereof will be omitted. This embodiment will describe a function corresponding to the tab 1103, and is applicable in combination of at least one of the above-described first and second embodiments, as a matter of course.

Personal Information UI

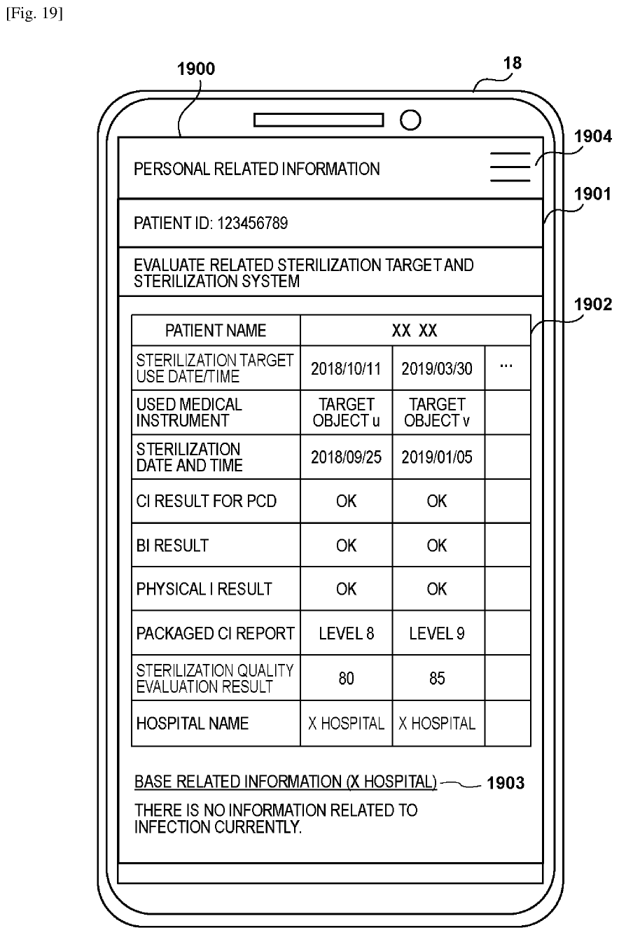

FIG. 19 shows an example of a UI of personal information provided from an evaluation analysis server 16 to a user terminal 18 according to this embodiment. A UI 1900 shown in FIG. 19 is displayed on the display unit of the user terminal 18, that is, a touch panel liquid crystal display of a smartphone or the like. However, the UI 1900 can be displayed on the display unit of a base server 14, a PC 15, or a user terminal 19 communicably connected to the evaluation analysis server 16 via a network 17 by adopting the output format of the UI 1900 to each apparatus. Since the UI 1900 is a user interface for displaying personal information, authentication of the user needs to be performed before the UI 1900 is displayed on the display unit of each apparatus. Control is executed so that personal information can be provided in accordance with the access level of the user obtained as a result of the authentication. If, for example, the user is a patient as a general user, only information related to himself/herself can be browsed. Note that although depending on the specifications of the system, the individual evaluation report described in the first embodiment and the comparison evaluation report described in the second embodiment may be released to general users. This allows the user to use the report as a criterion to select a medical institution for treatment.

The UI 1900 can be formed by including patient identification information 1901, patient-related information 1902, base-related information 1903, and a menu 1904. The patient identification information 1901 is information for identifying an authenticated patient who has logged in to the system. In this example, a patient ID is displayed. The patient-related information 1902 includes, for example, a patient name and information about a sterilization target used. The information about the sterilization target includes a use date/time, a sterilization date/time, the of a PCD CI, the result of a BI, the result of a physical indicator, the result of a CI in a package, a sterilization quality evaluation result, and a hospital name. As shown in FIG. 19, the information included in the patient-related information 1902 can be displayed for each sterilization target used. If the information cannot be displayed in one frame, it is displayed to be scrolled. For example, the user can scroll the patient-related information 1902 by a swipe operation, a flick operation, or the like on the touch panel liquid crystal display of the user terminal 18. These are merely examples, and are not intended to limit the present invention. The base-related information 1903 is a region where various kinds of information of a hospital where the patient undergoes treatment are displayed, and for example, information about infection is displayed. The menu 1904 allows the user to select a service provided by the evaluation analysis server 16 or make advanced settings and the like by tapping a display portion, similar to, for example, a case in which one of the tabs 1101 to 1104 of the UI 1200 is operated.

Processing Procedure

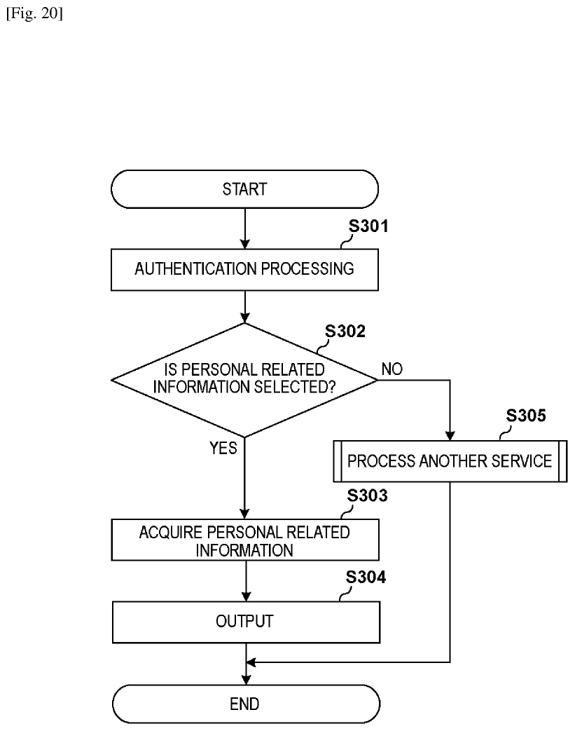

FIG. 20 is a flowchart illustrating a processing procedure when outputting the personal information according to this embodiment. As a use case of outputting the personal information, a case in which the user (patient) as a general user having the user terminal 18 accesses the evaluation analysis server 16 via the user terminal 18. Processing to be described below is implemented when, for example, a CPU 201 of the evaluation analysis server 16 reads out a control program stored in a ROM 203 or an HDD 204 into a RAM 202 and executes it. Note that this processing may be executed by each base server 14 or the user terminal 18 or 19. In this case, processing of acquiring necessary information from the evaluation analysis server 16 is added. A number following “S” to be described below indicates a step number in the flowchart.

In S301, the CPU 201 executes authentication processing of the user using a UI (not shown) in response to access from the user terminal 18. More specifically, the CPU 201 notifies the user terminal 18 of screen information about a login screen for requesting input of a patient ID and a password, and executes authentication processing of the user based on the information input via the login screen. Authentication of the user is performed with reference to user information held in a database 207. After authentication succeeds, the process advances to S302.

In S302, the CPU 201 displays, on the display unit of the user terminal 18, the UI 1900 in a state in which neither the patient-related information 1902 nor the base-related information 1903 is displayed, accepts an operation on the menu 1904, and determines whether acquisition of personal information has been requested. If acquisition of personal information has been requested, the process advances to S303; otherwise, the process advances to S305.

In S303, the CPU 201 acquires information related to a corresponding patient from the accumulation information in the database 207 using, as a search key, the patient ID acquired from the database 207. The CPU 201 acquires related information of the base used by the patient, for example, the base-related information of the base. In S304, the CPU 201 generates, for example, display output of the patient-related information 1902 and the base-related information 1903 of the UI 1900 based on the information acquired in S303, and transmits and outputs the display output to the user terminal 18 via the network 17, thereby ending the processing.

On the other hand, if another request is determined in S302, the CPU 201 executes, in S305, corresponding processing, for example, the processing associated with output of the individual evaluation report or the comparison evaluation report described in the first or second embodiment, thereby ending the processing.

As described above, if the user of the system according to this embodiment requests personal information, the system provides, to the user, an evaluation result concerning the sterilization target used. Furthermore, the system provides an evaluation result concerning the base used by the user. As described above, it is possible to release the information to a general user and the user (patient) can obtain information of a sterilization target used for himself/herself. Note that the individual evaluation report or the comparison evaluation report described in each of the above embodiments may be provided to a general user in accordance with his/her access level. In this case, evaluation items that can be provided may be restricted in accordance with the access level. By releasing such information to a general user, the patient can use the information as a criterion to select a medical institution to use.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2019-131399, filed Jul. 16, 2019, which is hereby incorporated by reference herein in its entirety.

1. A system for evaluating quality of a sterilization workflow concerning sterilization of a sterilization target,
the sterilization workflow having a plurality of stages including at least one of an assembly stage of grouping at least one sterilization target, a packaging stage of forming a set by packaging the at least one sterilization target, a sterilization stage of performing sterilization processing of the set by a sterilization processing apparatus, a storage stage of storing the set having undergone the sterilization processing in a predetermined storage place, a delivery stage of moving the set from the predetermined storage place to a place where the set is used, a use stage of using the at least one sterilization target included in the set by determining an achievement degree of the sterilization processing for the set, a collection stage of collecting the sterilization target used, and a cleaning stage of cleaning the collected sterilization target,
the system comprising:
a registration unit configured to register, for each base, basic information concerning the base where the sterilization workflow is performed;
an accumulation unit configured to acquire, for each base, accumulation information indicating a result of a work associated with the sterilization workflow, and accumulate the accumulation information; and
an evaluation unit configured to evaluate, in accordance with an evaluation target and an evaluation item designated by a user of the system, at least one evaluation item concerning at least one stage of the sterilization workflow using basic information concerning the evaluation target and the accumulation information.

2. The system according to claim 1, further comprising a providing unit configured to visualize and provide an evaluation result of the evaluation unit.

3. The system according to claim 2, wherein the evaluation unit performs an evaluation by classifying contents of a parameter associated with the evaluation item stepwise and scoring the contents.

4. The system according to claim 3, wherein the evaluation unit performs an evaluation using an evaluation table that defines, for each evaluation item, contents of a related parameter by classifying the contents stepwise and linking scores with the contents.

5. The system according to claim 3, wherein the evaluation unit further performs a comprehensive evaluation of the base by obtaining a weighted average of the score of at least one evaluated evaluation item in accordance with importance of each evaluation item.

6. The system according to claim 5, wherein the evaluation unit obtains the weighted average using a weighting table that registers, for each evaluation item, a weight corresponding to the importance in linkage with the evaluation item.

7. The system according to claim 6, wherein in the weighting table, the weight of each evaluation item can be changed in accordance with occurrence of infection.

8. The system according to claim 2, wherein the evaluation unit performs an evaluation of each evaluation item for every predetermined period.

9. The system according to claim 2, wherein the providing unit provides, in time series for every predetermined period, the evaluation result of at least one evaluation item designated by the user of the system.

10. The system according to claim 9, wherein the providing unit displays, on a display of an apparatus used by the user, a first user interface for displaying the evaluation result.

11. The system according to claim 10, wherein in the first user interface, a display format of the evaluation result can be switched to at least one of a table format, a line chart, and a radar chart.

12. The system according to claim 10, wherein in the first user interface, the predetermined period of the evaluation result displayed in time series for every predetermined period can be switched.

13. The system according to claim 10, wherein in the first user interface, the evaluation result displayed for each of the at least one evaluation item designated by the user of the system can be switched to a display format by at least one stage included in the sterilization workflow.

14. The system according to claim 13, wherein when switching to the display format by at least one stage included in the sterilization workflow, the providing unit acquires an evaluation result for each stage using the evaluation result of at least one evaluation item associated with each stage.

15. The system according to claim 2, wherein if there is lacking information for evaluating the at least one evaluation item designated by the user of the system, the evaluation unit requests the user for the lacking information.

16. The system according to claim 2, wherein if there is lacking information for evaluating the at least one evaluation item designated by the user of the system, the evaluation unit acquires the accumulation information of another base similar to the evaluation target among other bases different from the evaluation target, and alternatively uses the acquired accumulation information.

17. The system according to claim 2, wherein at least one of a predetermined base, a predetermined sterilization agent, a predetermined sterilization processing apparatus, a predetermined sterilization processing apparatus maintenance company, a predetermined indicator manufacturer, a predetermined outpatient, a predetermined inpatient, and a predetermined surgery patient can be designated as the evaluation target.

18. The system according to claim 2, wherein if the user of the system designates a plurality of comparison targets, the providing unit provides evaluation results of the plurality of comparison targets so as to compare the evaluation results.

19. The system according to claim 2, wherein if the user of the system designates similarity with a predetermined comparison target, the providing unit provides an evaluation result of the predetermined comparison target and an evaluation result of another comparison target whose degree of similarity with the predetermined comparison target exceeds the designated similarity so as to compare the evaluation results.

20. The system according to claim 19, wherein the similarity between the comparison targets is acquired using a degree of similarity of at least one item included in basic information between the comparison targets.

21. The system according to claim 18, wherein the providing unit displays, on the display of the apparatus used by the user, a second user interface for displaying the evaluation results of the plurality comparison targets so as to compare the evaluation results.

22. The system according to claim 21, wherein in the second user interface, the evaluation results of the respective comparison targets can be sorted in accordance with the evaluation result of one of the displayed evaluation items so as to compare the evaluation results.

23. The system according to claim 18, wherein the comparison targets is evaluated in at least one of a base unit, a sterilization agent unit, a sterilization processing apparatus unit, a sterilization processing apparatus maintenance company unit, a indicator manufacturer unit, a outpatient unit, a inpatient unit, and a surgery patient unit.

24. The system according to claim 2, wherein if the user of the system requests personal information, the providing unit provides an evaluation result concerning the sterilization target used for the user.

25. The system according to claim 24, wherein if the user of the system requests personal information, the providing unit further provides an evaluation result concerning a base used by the user.

26. The system according to claim 2, further comprising an authentication unit configured to authenticate the user,
wherein the providing unit restricts, in accordance with an access level of the user, information to be provided.

27. The system according to claim 1, wherein to determine the achievement degree of the sterilization processing in the sterilization workflow, at least one of a chemical indicator, a biological indicator, a physical indicator, and a cleaning indicator is used.

28. An information processing apparatus for evaluating quality of a sterilization workflow concerning sterilization of a sterilization target,
the sterilization workflow having a plurality of stages including at least one of an assembly stage of grouping at least one sterilization target, a packaging stage of forming a set by packaging the at least one sterilization target, a sterilization stage of performing sterilization processing of the set by a sterilization processing apparatus, a storage stage of storing the set having undergone the sterilization processing in a predetermined storage place, a delivery stage of moving the set from the predetermined storage place to a place where the set is used, a use stage of using the at least one sterilization target included in the set by determining an achievement degree of the sterilization processing for the set, a collection stage of collecting the sterilization target used, and a cleaning stage of cleaning the collected sterilization target,
the apparatus comprising:
a registration unit configured to acquire, for each base, from an external apparatus, basic information concerning the base where the sterilization workflow is performed, and register the acquired basic information;
an accumulation unit configured to acquire, for each base, from an external apparatus, accumulation information indicating a result of a work associated with the sterilization workflow, and accumulate the acquired accumulation information;
an evaluation unit configured to evaluate, in accordance with an evaluation target and an evaluation item designated by a user via an external apparatus, at least one evaluation item concerning at least one stage of the sterilization workflow using basic information concerning the evaluation target and the accumulation information; and
a providing unit configured to provide information for visualizing an evaluation result of the evaluation unit to an external apparatus used by the user.

29. A control method for an information processing apparatus for evaluating quality of a sterilization workflow concerning sterilization of a sterilization target,
the sterilization workflow having a plurality of stages including at least one of an assembly stage of grouping at least one sterilization target, a packaging stage of forming a set by packaging the at least one sterilization target, a sterilization stage of performing sterilization processing of the set by a sterilization processing apparatus, a storage stage of storing the set having undergone the sterilization processing in a predetermined storage place, a delivery stage of moving the set from the predetermined storage place to a place where the set is used, a use stage of using the at least one sterilization target included in the set by determining an achievement degree of the sterilization processing for the set, a collection stage of collecting the sterilization target used, and a cleaning stage of cleaning the collected sterilization target,
the method comprising:
acquiring, for each base, from an external apparatus, basic information concerning the base where the sterilization workflow is performed, and registering the acquired basic information;
acquiring, for each base, from an external apparatus, accumulation information indicating a result of a work associated with the sterilization workflow, and accumulating the acquired accumulation information;
evaluating, in accordance with an evaluation target and an evaluation item designated by a user via an external apparatus, at least one evaluation item concerning at least one stage of the sterilization workflow using basic information concerning the evaluation target and the accumulation information; and
providing information for visualizing an evaluation result of the evaluation to an external apparatus used by the user.

30. A non-transitory computer-readable storage medium storing a computer program for causing a computer to execute each step of a control method for an information processing apparatus for evaluating quality of a sterilization workflow concerning sterilization of a sterilization target,
the sterilization workflow having a plurality of stages including at least one of an assembly stage of grouping at least one sterilization target, a packaging stage of forming a set by packaging the at least one sterilization target, a sterilization stage of performing sterilization processing of the set by a sterilization processing apparatus, a storage stage of storing the set having undergone the sterilization processing in a predetermined storage place, a delivery stage of moving the set from the predetermined storage place to a place where the set is used, a use stage of using the at least one sterilization target included in the set by determining an achievement degree of the sterilization processing for the set, a collection stage of collecting the sterilization target used, and a cleaning stage of cleaning the collected sterilization target,
the method comprising:
acquiring, for each base, from an external apparatus, basic information concerning the base where the sterilization workflow is performed, and registering the acquired basic information;
acquiring, for each base, from an external apparatus, accumulation information indicating a result of a work associated with the sterilization workflow, and accumulating the acquired accumulation information;
evaluating, in accordance with an evaluation target and an evaluation item designated by a user via an external apparatus, at least one evaluation item concerning at least one stage of the sterilization workflow using basic information concerning the evaluation target and the accumulation information; and
providing information for visualizing an evaluation result of the evaluation to an external apparatus used by the user.

31. A system comprising a server configured to evaluate quality of a sterilization workflow concerning sterilization of a sterilization target and a user terminal configured to use a service provided by the server,
the sterilization workflow having a plurality of stages including at least one of an assembly stage of grouping at least one sterilization target, a packaging stage of forming a set by packaging the at least one sterilization target, a sterilization stage of performing sterilization processing of the set by a sterilization processing apparatus, a storage stage of storing the set having undergone the sterilization processing in a predetermined storage place, a delivery stage of moving the set from the predetermined storage place to a place where the set is used, a use stage of using the at least one sterilization target included in the set by determining an achievement degree of the sterilization processing for the set, a collection stage of collecting the sterilization target used, and a cleaning stage of cleaning the collected sterilization target,
the server comprising
a registration unit configured to acquire, for each base, from an external apparatus, basic information concerning the base where the sterilization workflow is performed, and register the acquired basic information,
an accumulation unit configured to acquire, for each base, from an external apparatus, accumulation information indicating a result of a work associated with the sterilization workflow, and accumulate the acquired accumulation information,
an evaluation unit configured to evaluate, in accordance with an evaluation target and an evaluation item designated by a user via the user terminal, at least one evaluation item concerning at least one stage of the sterilization workflow using basic information concerning the evaluation target and the accumulation information, and
a providing means for providing unit configured to provide information for visualizing an evaluation result of the evaluation unit to the user terminal, and
the user terminal comprising
a designation unit configured to designate the evaluation target and the evaluation item, and
a display unit configured to display, on a display, the information provided by the providing unit.

32. An information processing apparatus comprising:
input unit configured to input an evaluation target for which at least one evaluation item concerning at least one stage of a sterilization workflow is evaluated;
a selection unit configured to select at least one evaluation item that can be evaluated with respect to the evaluation target input via the input unit; and
a display control unit configured to display, on a display, an evaluation result of the at least one evaluation item selected via the selection unit.
